(19) United States
(12) Patent Application Publication
(10) Pub. No.: US 2026/0098247 A1
(43) Pub. Date: Apr. 9, 2026

(54) DEVELOPMENT OF SOLID-STATE STORAGE OF COMPLEX BIOCHEMICAL SYSTEMS

(71) Applicant: Cal Poly Corporation, San Luis Obispo, CA (US)

(72) Inventor: Javin Oza, San Luis Obispo, CA (US)

(21) Appl. No.: 19/114,264

(22) PCT Filed: Sep. 22, 2023
(86) PCT No.: PCT/US2023/074892

§ 371 Date: Mar. 21, 2025

Related U.S. Application Data
(60) Provisional application No. 63/376,857, filed on Sep. 23, 2022.

(51) Int. Cl. C12N 5/00; B01L 3/00

(52) U.S. Cl. CPC C12N 5/525; B01L 3/502707; B01L 2300/0848; B01L 2300/087; B01L 2300/0887

(57) ABSTRACT

Compositions for solid-state storage of a biological system and methods of making and using the same are described. The compositions comprise a polymeric cryoprotectant, a saccharide cryoprotectant, an emulsifier, and at least one lubricating flow agent, and are combined with a biological system to store the biological system in a solid-state with or without cold-chain infrastructure. The stabilized biological systems are suitable for use with one or more analytical devices to provide a cold chain-free, point-of-care, and low-cost platform for diagnostics, research, biomanufacturing, and testing.

### TECHNICAL FIELD

The present disclosure generally relates to compositions for solid-state storage of biological and chemical systems and components. The present disclosure particularly relates to solid-state compositions that are suitable for storage at temperatures ranging from −80° C. to 65° C. that readily rehydrate and activate the contained biological and chemical systems. The solid-state composition is suitable for use with analytical devices and may be incorporated into an array of reaction formats and applications.

### TECHNICAL BACKGROUND

Effective transportation and storage are critical for maintaining the integrity of sensitive biochemical systems. There are a wide variety of biochemical systems requiring global transportation and storage, including those comprising components such as genetic material, crude cell extracts comprising molecular machinery, energy molecules, salts, cofactors, excipients, and other complex molecules. However, technologies utilizing biochemical systems are often temperature-sensitive, relying on cold storage and transportation infrastructure in the form of refrigeration or freezing. This infrastructure, commonly referred to as the cold chain, involves the transportation of temperature-sensitive products along a supply chain using refrigerated, frozen, and other thermal packaging methods to enable long-term storage, transportation, and point-of-care biochemical applications. Cold chains often require an uninterrupted series of thermally controlled transportation and storage activities. Thus, the cold chain distribution process necessitates an expensive and complex supply chain network.

The dependence of the bioeconomy on the cold chain creates significant challenges for infrastructure, supply chain logistics, and costs. Further, the temperature control requirements in a cold chain limit access and distribution to key regions of the world. The Biotechnology Industry Organization has estimated that biological components will be one of the fastest growing sectors impacting the cold chain, with a market growth of approximately 15% per year.

Dependence on the cold chain limits the potential for emerging biotechnologies, restricting on-demand and point-of-care deployment due to prohibitive costs or the absence of infrastructure. Further, even when cold chain infrastructure can be leveraged for on-demand deployment, the environments where these technologies are used are often uncontrolled, which further limits the effectiveness of the technologies. Beyond on-demand deployment, breakdowns in the cold chain often result in reduced efficacy of pharmaceuticals. In the case of vaccines, cold chain breakdowns cost the pharmaceutical industry billions of dollars annually.

Cell-free protein synthesis (CFPS) is one example of a complex biochemical system where room temperature storage and stabilization would be extremely valuable. Cell-free expression systems utilizing CFPS have found widespread use for industrial and research applications because of their advantages over in vivo systems, namely in their capacity for plug-and-play of reaction components and ability to supersede homeostatic considerations. In the pharmaceutical industry, cell-free systems are being leveraged for both scaled-up production of clinically interesting targets as well as on-demand production of therapeutics at the point of care. As biosensors, cell-free systems have been configured to a number of analytes utilizing a variety of testing schemes and are especially useful for environmental biosensing where they mitigate contamination concerns associated with engineered microbes. As educational tools, cell-free systems have seen use in classroom modules showcasing the processes of transcription and translation to students, as well as more complex biotechnological processes like CRISPR/Cas. Beyond these applications, cell-free systems have also found uses in the biomanufacturing of consumer products like cosmetics and food goods, as well as in research applications such as the expression of proteins normally considered difficult-to-express in vivo and the prototyping of novel biotechnological systems by utilizing the open environment of cell-free platforms. Taken cumulatively, cell-free systems have demonstrated advantages in a broad spectrum of applications. Despite this, barriers of stability, storage, and proper application of these systems limit their effectiveness in the diverse range of settings where they could otherwise be useful.

Lyophilization has been presented as a potential solution to the problems caused by the cold chain. Multiple lyophilization strategies have been demonstrated to stabilize biochemical components for extended periods of time at room temperature. Accordingly, simple biochemical storage systems have been developed using lyophilization. However, stabilization using lyophilization has in the past occurred at the cost of biochemical system integrity. Lyophilization has been associated with a decrease in the activity of biochemical systems stemming from the inherent fragility of these reaction components. One proposed solution to this problem is the integration of cryoprotectant excipients, which have been observed to improve the activity of lyophilized systems. Platforms utilizing these techniques have demonstrated the ability to support simple enzymatic reactions and colorimetric dyes that are used in tandem with paper-based devices. However, such systems do not constitute a universal platform and fail to demonstrate wide applicability to a variety of complex biochemical systems.

There is therefore a need to develop storage systems for complex biochemical systems and components which are not dependent on a cold chain. More particularly, there is a need to develop storage systems for complex biochemical systems, such as CFPS, which are not dependent on a cold chain.

There is an additional need to develop a universal platform for the stabilization and storage of complex biochemical systems and components.

Relatedly, there is a need to develop such storage systems that maintain a high degree of system integrity, i.e., a storage system that results in minimal degradation.

There is a still further need to develop storage systems for complex biochemical systems and components which are also cost-effective.

These and other objects, advantages, and features of the present disclosure will become apparent from the following specification taken in conjunction with the claims set forth herein.

### BRIEF SUMMARY

The present disclosure relates to compositions for solid-state storage comprising a polymeric cryoprotectant; a saccharide cryoprotectant; an emulsifier; and a lubricating flow agent. In an embodiment, the polymeric cryoprotectant comprises a polyethylene oxide polymer, vinylpyrrolidone polymer, polysaccharide, polyampholyte, poloxamer, methoxy polyethylene glycol acrylate, or a combination thereof. In a further embodiment, the polyethylene oxide polymer comprises polyethylene glycol (PEG) having between 200 and 80000 ethylene glycol residues. In an embodiment, the saccharide cryoprotectant comprises a sugar alcohol, monosaccharide, disaccharide, oligosaccharide, polysaccharide, or a combination thereof. In a further embodiment, the saccharide cryoprotectant comprises glucose, galactose, fructose, ribose, lactose, sucrose, maltose, lactulose, trehalose, melibiose, glycerol, sorbitol, mannitol, maltitol, a starch, dextrin, dextran, cellulose, methylcellulose, hemicellulose, pectin, gelatin, glycogen, chitin, heparin, maltodextrin, a sugar alcohol, or a combination thereof. In a still further embodiment, the saccharide cryoprotectant comprises trehalose dihydrate, anhydrous dextrose, anhydrous glucose, anhydrous crystalline maltose, or a combination thereof.

According to some embodiments, the emulsifier comprises microcrystalline cellulose, defibrillated microcrystalline cellulose, microfibrillated cellulose, powdered cellulose, cellulose, cellulose acetate, hydroxypropyl methylcellulose, carboxymethylcellulose, sodium carboxymethylcellulose, ethylcellulose, methylcellulose, hydroxyethylcellulose, hydroxyethylcellulose, hydroxypropylcellulose, silica nanoparticles, carbon nanotubes, or a combination thereof.

In an embodiment, the lubricating flow agent comprises a fatty acid metal salt, a fatty acid, hydrocarbon, fatty alcohol, fatty acid ester, alkyl sulfate, polymer, or a combination thereof. In a further embodiment, the fatty acid comprises oleic acid, butyric acid, lauric acid, palmitic acid, benzoic acid, fumaric acid, stearic acid, or a combination thereof; and wherein the counterion of the fatty acid is a metal or a metal salt comprising lithium, sodium, and potassium, magnesium, calcium, barium, lead, zinc, cadmium, or a combination thereof. In some embodiments, the fatty acid is conjugated to an organic compound, a glycerol, PEG, or a combination thereof. In some embodiments, the lubricating flow agent comprises a first lubricating flow agent and a second lubricating flow agent. According to an embodiment, the first lubricating flow agent comprises sodium stearyl fumarate, and wherein the second lubricating flow agent comprises magnesium stearate. In some embodiments, the flow agent or lubricating agent is a silicate. In some embodiments, the counterion is a monovalent or divalent cation.

In an embodiment, the composition comprises between about 0.5% to about 10% of the polymeric cryoprotectant, between about 1 mM to about 50 mM of the saccharide cryoprotectant, between about 0.1% to about 10% of the emulsifier; and between about 0.5% and about 20% of the lubricating flow agent.

In an embodiment, the solid-state storage material is coated. In an embodiment, a coating modifies the conditions for rehydration of solid-state materials. In another embodiment, the coating provides additional protection against specific environmental or storage conditions. The coating comprises natural and synthetic polymers containing acetate, phthalate, succinate, alginate, zein, saccharides, hypromellose, cellulose, chitin, or polyvinyl.

In an embodiment, solid-state storage formulation contains one or more preservatives. In a further embodiment, the preservative comprises a methyl paraben, ethyl paraben, propyl paraben and/or butyl paraben, sorbic acid, sodium sorbate, calcium sorbate, and/or potassium sorbate, benzoic acid, sodium benzoate, potassium benzoate, and/or calcium benzoate, sodium metabisulfite, propylene glycol, BHT, BHA, benzaldehyde, an essential oil, a phenol, or a combination thereof.

The present disclosure also relates to methods of stabilizing a biological system in solid-state storage comprising contacting a biological system with a composition comprising a polymeric cryoprotectant, a saccharide cryoprotectant, an emulsifier, and a lubricating flow agent to form a mixture, lyophilizing the mixture; and forming the mixture into a solid. In an embodiment, the solid is a powder, flowable powder, agglomerate, flake, granule, pellet, tablet, or pencil. In a further embodiment, the solid can be stored and transported a large range of temperatures (−80° C. to 65° C.) and retain at least 50% activity of the biological system.

In a further embodiment, the solid can withstand exposure to various environmental conditions or compounds such as organic compounds and solvents and/or humidity conditions ranging from 0% to 80% and retain at least 50% of activity of the biological system.

In some embodiments, the methods further comprise a step of depositing the solid onto a substrate. In an embodiment, the substrate is a microfluidic chip or a paper-based microfluidic device.

The disclosure also relates to methods of using a stabilized biological system comprising (i) obtaining a stabilized biological system comprising a biological system, a polymeric cryoprotectant, a saccharide cryoprotectant, an emulsifier, and a lubricating flow agent; wherein the stabilized biological system is a solid; (ii) optionally depositing the stabilized biological system onto a substrate; and (iii) contacting the stabilized biological system with a liquid to form a resuspension solution.

In some embodiments, the method further comprises (iv) collecting a biological sample; (v) reacting the resuspension solution with the biological sample to generate a result; and (vi) interpreting the result.

The present disclosure also relates to paper-based microfluidic devices comprising a porous substrate comprising paper; an input channel; a reaction chamber; one or more microfluidic channels; and a non-porous barrier that defines one or more boundaries for the reaction chamber and the one or more microfluidic channels; wherein the reaction chamber is fluidly connected to the input chamber by the one or more microfluidic channels; and wherein the surface of the microfluidic device is laminated except for the reaction chamber. In an embodiment, the substrate is paper-like material comprised of natural or synthetic materials, woven fibers, non-woven fibers, or textiles. In another embodiment, the device is wearable.

Further discussion of suitable textiles and wearable devices is found in Lu, Textile-Embedded Cell-Free Biosensors, NAT. BIOMED. ENG'G, 2022: 6, 225-226, along with Nguyen, et al., Wearable Materials With Embedded Synthetic Biological Sensors for Biomolecule Detection, NAT. BIOTECH. 2021: 39: 1366-1374, which are both herein incorporated by reference in their entirety.

While multiple embodiments are disclosed, still other embodiments of the present disclosure will become apparent based on the detailed description, which shows and describes illustrative embodiments of the disclosure. The foregoing summary is illustrative only and is not intended to be in any way limiting. In addition to the illustrative aspects, embodiments, and features described above, further aspects, embodiments, and features of the present technology are apparent from the following drawings and the detailed description, which shows and describes illustrative embodiments of the present technology. Each feature of the technology described herein may be combined with any one or more other features of the disclosure, e.g., the methods may be used with any composition described herein. Accordingly, the drawings and detailed description are to be regarded as illustrative and not restrictive.

### BRIEF DESCRIPTION OF THE DRAWINGS

FIG. 1 shows an example method for stabilizing a CFPS system. In particular, FIG. 1 shows the simplified steps toward integrating cell-free expression into a pellet platform, wherein the components of a cell-free reaction are mixed to start a CFPS reaction. This reaction mix can run an aqueous CFPS reaction expressing a target construct via incubation or be lyophilized. This FDCF material can be rehydrated to start the CFPS reaction or be pressed into a pellet for a range of applications, including in diagnostic testing, room-temperature solid-state storage, and portable, on-demand biosynthesis.

FIG. 2A shows the results of a lyophilized CFPS test demonstrating the effect of lyophilization on CFPS activity.

FIG. 2B shows the results of a lyophilized CFPS test demonstrating that increasing lyophilization time greatly improves CFPS activity recovery.

FIG. 2C shows the results of a lyophilized CFPS test demonstrating the integration of energy and cofactor reagents into the lyophilized mix and the impact of CFPS activity.

FIG. 2D shows the results of a lyophilized CFPS test and how scaling up the reaction volume negatively impacts CFPS activity.

FIG. 3A shows the results of adding cryoprotective excipients (0.4% PEG8000; 20 mM Trehalose Dihydrate) to recover expression of pJL1-sfGFP in lyophilized 75 uL CFPS reactions.

FIG. 3B shows the result of adding cryoprotective excipients observed to recover expression in scaling volume of CFPS reaction to 300 uL, even with the addition of 5% graphite.

FIG. 3C is an image that demonstrates the utilization of experimental techniques to recover expression of pJL1-sfGFP through increasing oxygen availability, reaction surface area, and sfGFP post-translational folding.

FIG. 3D is an image that demonstrates the utilization of experimental techniques to recover expression of pJL1-sfGFP through increasing oxygen availability, reaction surface area, and sfGFP post-translational folding.

FIG. 3E is an image that demonstrates the utilization of experimental techniques to recover expression of pJL1-sfGFP through increasing oxygen availability, reaction surface area, and sfGFP post-translational folding

FIG. 3F depicts the quantified results of the images of FIG. 3A, wherein CFPS activity was validated at 800 uL, the reaction volume necessary for pellet fabrication.

FIG. 4A shows the results of additive titrations in CFPS, specifically a graphite titration at 5%-25%.

FIG. 4B shows the results of additive titrations in CFPS, specifically a PEG2000 titration at 5%-25%.

FIG. 4C shows the results of additive titrations in CFPS, specifically a maltodextrin titration at 0.5%-5%.

FIG. 4D shows the results of additive titrations in CFPS, specifically an MCC titration at 0.5%-10%.

FIG. 4E shows the results of additive titrations in CFPS, specifically an Mg2S titration at 2%-10%.

FIG. 4F shows the results of additive titrations in CFPS, specifically an SSF titration at 2%-10%.

FIG. 5A shows an image of the reaction of HRP/Gox reagent pencil pellets.

FIG. 5B shows an image of the reaction of HRP/Gox reagent pencil pellets.

FIG. 5C shows an image of the qualitative results of the successful expression of pJL1-sfGFP on a μPAD via pellet deposition.

FIG. 5D shows an image of the qualitative results of the successful expression of pJL1-sfGFP on a μPAD via pellet deposition.

FIG. 5E is a graph of the resuspension of pJL1-sfGFP pellets integrating energy and cofactor reagents in the lyophilization mixture.

FIG. 6A depicts testing material metrics of additives in pellet deposition and resuspension, in particular the PEG2000 deposition titration test.

FIG. 6B depicts testing material metrics of additives in pellet deposition and resuspension, in particular the MCC deposition titration test.

FIG. 6C depicts testing material metrics of additives in pellet deposition and resuspension, in particular the SSF resuspension titration test.

FIG. 6D depicts testing material metrics of additives in pellet deposition and resuspension, in particular the Mg2S resuspension titration test.

FIG. 7A depicts the pellet manufacturing process, beginning with the CFPS mixture before and after the lyophilization process, then the process of pressing the lyophilized material into a cohesive pellet.

FIG. 7B depicts the differences between lyophilized material with (left) and without (right) energy and cofactor reagents, wherein the left material was observed to be able to be pressed into a cohesive pellet while the right material was observed to be too brittle to form a cohesive pellet.

FIG. 7C depicts the segmentation of the pellet via a scalpel., measurement of diameter, length, and mass of the segments to decrease error in resuspension, and lastly resuspension and pJL1-sfGFP expression of the pellet segments.

FIG. 7D depicts the measurement of diameter, length, and mass of the segments of the pellet to decrease error in resuspension.

FIG. 7E shows the resuspension and pJL1-sfGFP expression of the pellet segments.

FIG. 8A shows the process of prototyping the integration of excipients for pellet resuspension and expression, in particular showing resuspended pellets before and after one round of 10-second pulse vortexing, highlighting differences in pellet excipient treatments'capacity for resuspension.

FIG. 8B shows the process of prototyping the integration of excipients for pellet resuspension and expression, in particular, expression after 14 hr. incubation at 37° C. of 4a pellets, highlighting differences in pellet capacity for pJL1-sfGFP expression after resuspension.

FIG. 8C shows photographs of the process of prototyping the integration of excipients for pellet resuspension and expression.

FIG. 8D shows the process of prototyping the integration of excipients for pellet resuspension and expression, in particular, the replicate expression test observing pJL1-sfGFP expression of pellets segmented into three pellet segments and individually resuspended and incubated, highlighting the homogeneity of CFPS reagents integrated into the pellet platform.

FIG. 8E shows photographs of the process of prototyping the integration of excipients for pellet resuspension and expression.

FIG. 9A depicts the prototyping of pJL1-sfGFP expression in pellets with integrated additives, in particular, the μPAD pellet+additives deposition expression test.

FIG. 9B shows images of the prototyping of pJL1-sfGFP expression in pellets with integrated additives, in particular, the μPAD pellet+additives deposition expression test.

FIG. 9C depicts the prototyping of pJL1-sfGFP expression in pellets with integrated additives, in particular, the resuspension of pellet segment+additives after 5 and 10-second cycles of pulse vortexing.

FIG. 9D depicts the prototyping of pJL1-sfGFP expression in pellets with integrated additives, in particular, the μPAD pellet+additives deposition expression test.

FIG. 9E shows the pellet expression time series over 15 minutes to 4 hours, in 15-minute intervals.

FIG. 10A shows visual observations of the resuspension of pellet segments containing combinations of excipients. This Figure highlights differences in the excipient combinations' capacity to optimize pellet resuspension.

FIG. 10B is a graph quantifying pJL1-sfGFP expression after pellet segment resuspension and incubation.

FIG. 10C shows visual observations of the capacity for pellet segments containing excipient combinations to successfully express pJL1-sfGFP after resuspension.

FIG. 11A depicts the aqueous deposition and reaction of mRFP1 and CRISPR-SpCas9 System on μPADs as part of prototyping the CRISPR-SpCas9 system in the pellet platform.

FIG. 11B shows images of the aqueous deposition and reaction of mRFP1 and CRISPR-SpCas9 System on μPADs as part of prototyping the CRISPR-SpCas9 system in the pellet platform.

FIG. 11C shows the results of the pellet+CRISPR-SpCas9 resuspension test of FIG. 11A.

FIG. 12A is a graph of SpCas9 titration demonstrating the clouding effect of increasing SpCas9 concentration. Without being bound by theory, it is thought that this is the result of nonspecific endonuclease activity.

FIG. 12B is a combination of a graph and images demonstrating the successful integration and expression of pY71-mRFP1 in the pellet platform, which is key for testing the expression and activity of the CRISPR-SpCas9 system in pellets.

FIG. 12C shows a graph demonstrating successful CRISPR-SpCas9 expression and activity in the pellet platform, wherein the quenching of pY71-mRFP1 expression in the gRNA+ treatments by the CRISPR system is clearly visible on the pellet-deposited μPADs.

FIG. 12D shows images demonstrating successful CRISPR-SpCas9 expression and activity in the pellet platform, wherein the quenching of pY71-mRFP1 expression in the gRNA+ treatments by the CRISPR system is clearly visible on the pellet-deposited μPADs.

FIG. 13A shows images demonstrating the evaporation of CFPS reaction solution and attempts to solve this problem via lamination.

FIG. 13B is an image of a laminated μPAD after incubation in an Eppendorf tube with 50 μL of water to increase vapor pressure.

FIG. 13C is a graph demonstrating the successful pJL1-sfGFP expression via pellet deposition on μPADs.

FIG. 15D shows images demonstrating the successful pJL1-sfGFP expression via pellet deposition on μPADs.

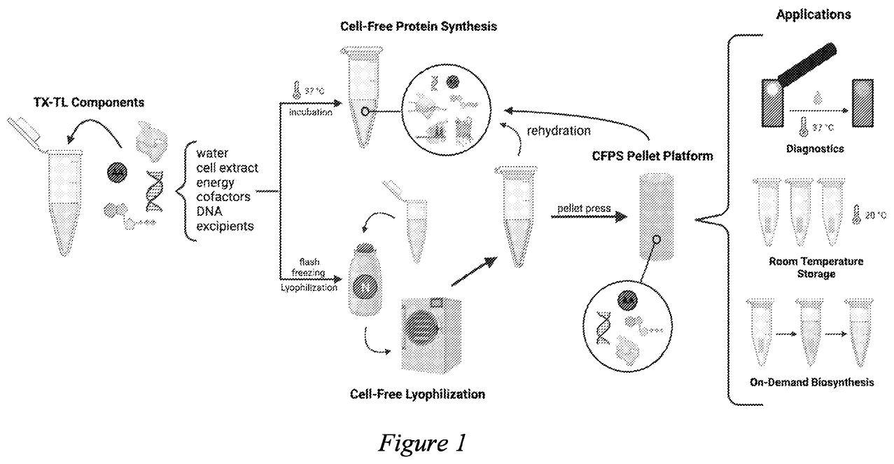

FIG. 14A shows the aqueous deposition of CFPS reactions+PEG2000 titration onto μPADs (UV imaging).

FIG. 14B shows the Pellet+graphite+PEG2000 material deposition on μPADs (UV imaging).

FIG. 14C shows a successful μPADs removing test channel for well-only CFPS reactions.

FIG. 14D is an image of pellet-deposited μPADs pJL1-sfGFP expression tests, wherein some leakage onto parafilm was observed.

FIG. 14E is a graph of pellet-deposited μPADs pJL1-sfGFP expression tests.

Various embodiments of the present disclosure will be described in detail regarding the drawings. Reference to various embodiments does not limit the scope of the disclosure. Figures represented herein are not limitations on the various embodiments according to the disclosure and are presented as an example illustration of the disclosure.

### DETAILED DESCRIPTION OF THE PREFERRED EMBODIMENTS

The present disclosure relates to solid-state platforms that can be used to provide effective storage, transportation, and on-demand usage of complex biochemical systems. This platform comprises a mixture of lyophilized biochemical reagents manufactured into easily portable and stable solids, such as cylindrical pellets. Beneficially, these solids (e.g., cylindrical pellets) may be resuspended in water for either on-demand biochemical reactions or use with paper-based devices. Whether resuspended or incorporated into paper, the disclosed platforms and systems beneficially provide effective diagnostics at the point of care.

In a preferred embodiment, the solid-state platforms rely on Cell-Free Protein Synthesis (CFPS) as an example of complex biochemical systems supported by the platforms. CFPS is an excellent model for many types of complex biochemical systems, particularly because it generally uses biological machinery to express proteins from a genetic template without living cells. In a still further preferred embodiment, the solid-state platforms stabilize a CFPS system relating to the expression of pJL1-sfGFP. In a further preferred embodiment, the solid-state platforms stabilize a CFPS system relating to the expression and activity of the CRISPR/SpCas9 system.

Beneficially, the solid-state platforms described herein not only stabilize complex biochemical systems at room temperature (20-22° C., 68-72° F.) but also increase these systems' capacity for resuspension in solution and deposition onto μPADs (Paper-Based Microfluidic Devices) without a detrimental decrease in CFPS reactions.

The embodiments of this disclosure are not limited to particular types of compositions or methods, which can vary. It is further to be understood that all terminology used herein is to describe particular embodiments only and is not intended to be limiting in any manner or scope. For example, as used in this specification and the appended claims, the singular forms “a,” “ ” “an” and “the” can include plural referents unless the context indicates otherwise. Unless indicated otherwise, “or” can mean any one alone or any combination thereof, e.g., “A, B, or C” means the same as any of A alone, B alone, C alone, “A and B,” “A and C,” “B and C” or “A, B, and C.” Further, all units, prefixes, and symbols may be denoted in its SI accepted form.

As used herein, the terms “comprise,” comprises,“ comprising,” “include,” “includes,” and “including” can be interchanged and are to be construed as at least having the features to which they refer while not excluding any additional unspecified features.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, a description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6, and decimals and fractions, for example, 1.2, 3.8, 1½, and 4¾ This applies regardless of the breadth of the range.

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

The terms “a,” “an,” and “the” include both singular and plural referents.

The term “or” is synonymous with “and/or” and means any one member or combination of members of a particular list.

The term “about,” as used herein, refers to variation in the numerical quantity that can occur, for example, through typical measuring techniques and equipment, with respect to any quantifiable variable, including, but not limited to, mass, volume, time, temperature, pH, reflectance, whiteness, etc. Further, in practical handling procedures, there is certain inadvertent error and variation that is likely through differences in the manufacture, source, or purity of the ingredients used to make the compositions or carry out the methods and the like. The term “about” also encompasses amounts that differ due to different equilibrium conditions for a composition resulting from a particular initial mixture. The term “about” also encompasses these variations. Whether or not modified by the term “about,” the claims include equivalents to the quantities.

The term “biological system” is used herein to refer to one or more synthetic or natural biological, chemical, biochemical, organic, or inorganic components in need of stabilization, storage, transportation, simplicity for deployment by the end-user. A biological system can include, without limitation, genetic material, enzymes, ribozymes, aptamers, riboswitches, reconstituted systems, artificial cells, crude cell extracts containing molecular machinery, energy molecules, salts, cofactors, excipients, reagents, a biological sample, or other complex molecules. The term biological system may encompass all components of a platform (e.g., CFPS, CRISPR-Cas9), a part of a platform (e.g., one or more reagents), or molecules or compounds. The term “biological system” is also used interchangeably with a “component in need of stabilizing” and a “component to be stabilized.”

The term “biomolecule” refers to any chemical species which derives its effect on living cells, systems, reactions, or organisms by virtue of molecular structures, including primary chemical structure and secondary chemical structures. As used herein, the term “biomolecule” refers to one or more biomolecules, and includes but is not limited to, the following: proteins, polypeptides, or complexes, analogs, or derivatives thereof; DNA, RNA, polynucleotides, or complexes, analogs, or derivatives thereof; plasmid vectors; final products, i.e., commercial embodiments or processed forms of the product; intermediate products, i.e., unprocessed products, e.g., prior to conversion to an active species, or denatured species; reagents or chemical compounds or elements, including those used to initiate or facilitate a desired reaction; peptide nucleic acids (PNA), monoclonal or polyclonal antibodies, derivatives of the commercial embodiment, i.e., antibodies specific to the commercial embodiment or chemically modified forms of the commercial embodiment; or fragments, complexes, or analogs of the commercial embodiment.

The methods, systems, apparatuses, and compositions disclosed herein may comprise, consist essentially of, or consist of the components and ingredients described herein as well as other ingredients not described herein. As used herein, “consisting essentially of” means that the methods, systems, apparatuses, and compositions may include additional steps, components, or ingredients, but only if the additional steps, components, or ingredients do not materially alter the basic and novel characteristics of the claimed methods, systems, apparatuses, and compositions.

It should also be noted that, as used in this specification and the appended claims, the term “configured” describes a system, apparatus, or other structure that is constructed or configured to perform a particular task or adopt a particular configuration. The term “configured” can be used interchangeably with other similar phrases such as arranged and configured, constructed and arranged, adapted and configured, adapted, constructed, manufactured and arranged, and the like.

The “scope” of the present disclosure is defined by the claims, along with the full scope of equivalents to which such claims are entitled. The scope of the disclosure is further qualified as including any possible modification to any of the aspects and/or embodiments disclosed herein which would result in other embodiments, combinations, sub-combinations, or the like that would be obvious to those skilled in the art.

The term “amplification reaction” refers to any chemical reaction, including an enzymatic reaction, which results in increased copies of a template nucleic acid sequence or results in transcription of a template nucleic acid. Amplification reactions include reverse transcription, the polymerase chain reaction (PCR), including Real-Time PCR (see U.S. Pat. Nos. 4,683,195 and 4,683,202; PCR Protocols: A Guide to Methods and Applications (Innis et al., eds, 1990)), and the ligase chain reaction (LCR) (see Barany et al., U.S. Pat. No. 5,494,810). For example, “amplification reactions conditions” or “amplification conditions” typically comprise either two-or three-step cycles. Two-step cycles have a high-temperature denaturation step followed by a hybridization/elongation (or ligation) step. Three-step cycles comprise a denaturation step followed by a hybridization step followed by a separate elongation step.

The terms “target,” “target sequence,” “target region,” and “target nucleic acid,” as used herein, are synonymous and refer to a region or sequence of a nucleic acid which is to be amplified, sequenced, or detected.

The term “primer,” as used herein, refers to an oligonucleotide capable of acting as a point of initiation of DNA synthesis under suitable conditions. Such conditions include those in which the synthesis of a primer extension product complementary to a nucleic acid strand is induced in the presence of four different nucleoside triphosphates and an agent for extension (for example, a DNA polymerase or reverse transcriptase) in an appropriate buffer and at a suitable temperature.

A primer is preferably a single-stranded DNA. The appropriate length of a primer depends on the intended use of the primer but typically ranges from about 6 to about 225 nucleotides, including intermediate ranges, such as from 15 to 35 nucleotides, from 18 to 75 nucleotides, and from 25 to 150 nucleotides. Short primer molecules generally require cooler temperatures to form sufficiently stable hybrid complexes with the template. A primer need not reflect the exact sequence of the template nucleic acid but must be sufficiently complementary to hybridize with the template. The design of suitable primers for the amplification of a given target sequence is well known in the art and described in the literature cited herein.

Primers can incorporate additional features which allow for the detection or immobilization of the primer but do not alter the basic property of the primer, that of acting as a point of initiation of DNA synthesis. For example, primers may contain an additional nucleic acid sequence at the 5′ end that does not hybridize to the target nucleic acid, but which facilitates cloning or detection of the amplified product, or enables the transcription of RNA (for example, by the inclusion of a promoter) or translation of protein (for example, by the inclusion of a 5′-UTR, such as an Internal Ribosome Entry Site (IRES) or a 3′-UTR element, such as a poly(A)ₙ sequence, where n is in the range from about 20 to about 200). The region of the primer that is sufficiently complementary to the template to hybridize is referred to herein as the hybridizing region.

As used herein, a primer is “specific,” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer, and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences that contain the target primer binding sites.

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase, and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, RNA polymerases of bacteriophages (e.g., T3 RNA polymerase, T7 RNA polymerase, SP6 RNA polymerase), and E. coli RNA polymerase, among others. The foregoing examples of RNA polymerases are also known as DNA-dependent RNA polymerases. The polymerase activity of any of the above enzymes can be determined by means well known in the art.

The term “promoter” refers to a cis-acting DNA sequence that directs RNA polymerase and other trans-acting transcription factors to initiate RNA transcription from the DNA template that includes the cis-acting DNA sequence.

As used herein, the term “sequence defined biopolymer” refers to a biopolymer having a specific primary sequence. A sequence-defined biopolymer can be equivalent to a genetically encoded defined biopolymer in cases where a gene encodes the biopolymer having a specific primary sequence. As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as a “gene product.” If the polynucleotide is derived from genomic DNA, expression may include splicing of the mRNA in a eukaryotic cell.

As used herein, “expression template” refers to a nucleic acid that serves as a substrate for transcribing at least one RNA that can be translated into a sequence-defined biopolymer (e.g., a polypeptide or protein). Expression templates include nucleic acids composed of DNA or RNA. Suitable sources of DNA for use as nucleic acids for an expression template include genomic DNA, cDNA, and RNA that can be converted into cDNA. Genomic DNA, cDNA, and RNA can be from any biological source, such as a tissue sample, a biopsy, a swab, sputum, a blood sample, a fecal sample, a urine sample, or a scraping, among others. The genomic DNA, cDNA, and RNA can be from a host cell or virus origins and any species. As used herein, “expression template” and “transcription template” have the same meaning and are used interchangeably.

In certain embodiments, vectors such as, for example, expression vectors containing a nucleic acid encoding one or more rRNAs or reporter polypeptides and/or proteins described herein are provided. As used herein, the term “vector” refers to a nucleic acid molecule capable of transporting another nucleic acid to which it has been linked. One type of vector is a “plasmid,” which refers to a circular double-stranded DNA loop into which additional DNA segments can be ligated. Such vectors are referred to herein as “expression vectors.” In general, expression vectors of utility in recombinant DNA techniques are often in the form of plasmids. In the present specification, “plasmid” and “vector” can be used interchangeably. However, the disclosed methods and compositions are intended to include such other forms of expression vectors, such as viral vectors (e.g., replication defective retroviruses, adenoviruses, and adeno-associated viruses), which serve equivalent functions.

In certain embodiments, the recombinant expression vectors comprise a nucleic acid sequence (e.g., a nucleic acid sequence encoding one or more rRNAs or reporter polypeptides and/or proteins described herein) in a form suitable for expression of the nucleic acid sequence in one or more of the methods described herein, which means that the recombinant expression vectors include one or more regulatory sequences that are operatively linked to the nucleic acid sequence to be expressed. Within a recombinant expression vector, “operably linked” is intended to mean that the nucleotide sequence encoding one or more rRNAs or reporter polypeptides and/or proteins described herein is linked to the regulatory sequence(s) in a manner that allows for the expression of the nucleotide sequence (e.g., in an in vitro ribosomal assembly, transcription and/or translation system). The term “regulatory sequence” is intended to include promoters, enhancers, and other expression control elements (e.g., polyadenylation signals).

The polynucleotide sequences contemplated herein may be present in expression vectors. For example, the vectors may comprise: (a) a polynucleotide encoding an ORF of a protein; (b) a polynucleotide that expresses an RNA that directs RNA-mediated binding, nicking, and/or cleaving of a target DNA sequence; and both (a) and (b). The polynucleotide present in the vector may be operably linked to a prokaryotic or eukaryotic promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary join two protein coding regions, in the same reading frame. Vectors contemplated herein may comprise a heterologous promoter (e.g., a eukaryotic or prokaryotic promoter) operably linked to a polynucleotide that encodes a protein. A “heterologous promoter” refers to a promoter that is not the native or endogenous promoter for the protein or RNA that is being expressed. Vectors as disclosed herein may include plasmid vectors.

As used herein, the terms “peptide,” “polypeptide,” and “protein,” refer to molecules comprising a chain a polymer of amino acid residues joined by amide linkages. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include nonstandard, noncanonical, or unnatural amino acids, which optionally may include amino acids other than any of the following amino acids: alanine, cysteine, aspartic acid, glutamic acid, phenylalanine, glycine, histidine, isoleucine, lysine, leucine, methionine, asparagine, proline, glutamine, arginine, serine, threonine, valine, tryptophan, and tyrosine residues. The term “amino acid residue” may include alpha-, beta-, gamma-, and delta-amino acids.

### Solid-state Storage Systems and Compositions

Complex biological systems exhibit sensitivity to traditional lyophilization methods, wherein the activity of the system is lost during resuspension. Although certain components, such as polyethylene glycol and trehalose dihydrate, have been identified as useful in partial preservation of lyophilized sample activity, the performance of these components alone is insufficient to preserve enough activity, particularly for scaled-up, point-of-care use. Beneficially, the methods described herein provide effective solid-state storage of complex biological systems with excellent recovery of system activity. Traditional lyophilization methods cannot be incorporated into solid-state storage systems without loss of expression. For example, the simple combination of a CFPS workflow with traditional lyophilization methods, wherein cell extract, a DNA template, energy source, and cofactors are combined, flash frozen, lyophilized, and resuspended, results in decreased expression in comparison to a non-lyophilized, aqueous CFPS workflow. Similarly, the simple scaling up of traditional lyophilization methods results in a loss of the biological system's activity. Without being bound by theory, it is thought that scaling up is unsuccessful due to issues such as the sensitivity of reaction components to lyophilization, limited oxygen availability upon rehydration, limited surface area for the reaction to take place, or a combination thereof.

### Polymeric Cryoprotectant

In an embodiment, the compositions comprise a polymeric cryoprotectant. The polymeric cryoprotectant prevents damage to the biological system or sample during lyophilization and may also provide secondary benefits by functioning as a stabilizing agent, coating agent, or more generally an excipient. Suitable polymers include, without limitation, polymers of ethylene oxide, vinylpyrrolidone, polysaccharides, mixed charged polymers (polyampholytes), or a combination thereof.

More particularly, suitable polymers include nonionic polymers such as polyethylene glycol (PEG), sorbitan monolaurate (Tween 20), polyoxyethylene sorbitan monooleate (Tween 80), a block co-polymer of polyethylene and polypropylene glycol (EO/PO copolymer) such as a Pluronic (EO_{n/2}-POₘ-EO_{n/2} copolymer) or a reverse Pluronic (PO_{n/2} -EOₘ-PO_{n/2} copolymer). When referring to PEG, the size of the PEG moieties (e.g., PEG8000) refers to the chain length, namely the number of ethylene glycol residues in the PEG chain. For example, PEG8000 has 8000 ethylene glycol residues in the PEG polymer and PEG2000 has 2000 ethylene glycol residues in the PEG polymer, etc. Suitable PEG polymers include a PEG polymer in the range of PEG200 to PEG80000, inclusive of all polymers within this range (e.g., PEG2000). In a preferred embodiment, the polymeric cryoprotectant is PEG2000 or PEG8000. Other suitable polymers include water-soluble, non-toxic polymers such as polyvinylpyrrolidone (PVP), human serum albumin (HSA), xanthan gum, carrageenan, collagen, chondroitin sulfate, a sialic acid polymer, or a combination thereof. Additionally, polyampholytes may be used as a polymeric cryoprotectant, including, for example, triblock copolymers of methacrylic acid (MAA) and 2-(dimethylamino)ethyl methacrylate (DMAEMA) and either methyl methacrylate or 2-phenylethyl methacrylate using group transfer polymerization, or polyampholytes based on substituted styrenes using nitroxide mediated polymerization, for example, COOH-εPLL, DMAEMA-MAA, DMAEMA-MAA-OcMA, Poly-CMB, Poly-SPB, MVE-MA(NH₂), Azide-Dex-PA, DMAEMA-MAA-BuMA, DMAPMA-MAA, DMAPMA-MAA-BuAAm, or a combination thereof.

The polymeric cryoprotectant may be present in the composition in an amount of between about 0.5% to about 10%, more preferably between about 1% and about 5%, still more preferably between about 2% and about 3%, inclusive of all integers within these ranges.

### Saccharide Cryoprotectant

The compositions preferable include a saccharide cryoprotectant such as a sugar alcohol, monosaccharide, disaccharide, oligosaccharide, polysaccharide, or a derivative, a salt, or a combination thereof. Suitable saccharides include, without limitation, monosaccharides such glucose, galactose, fructose, and ribose, disaccharides such as lactose, sucrose, lactulose, trehalose, and melibiose, and sugar alcohols such as glycerol, sorbitol, and mannitol, or any combination thereof. Polysaccharide polymers may also be used, including starch, dextrin, dextran, cellulose, methylcellulose, pectin, glycogen, or a derivative or a combination thereof. More particularly, suitable polysaccharides include hydroxyethyl starch (HES), gelatin, non-hydrolyzed gelatin, alginate, arabinoxylan, beta-glucan, cellulose, chitin, gellan, guar, inulin, lignin, pectin, xanthan, amylose, amylopectin, or a derivative or a combination thereof.

The saccharide cryoprotectant may be provided in a crystalline or anhydrous form, such as trehalose dihydrate, anhydrous dextrose, anhydrous glucose, anhydrous crystalline maltose, and the like. In a preferred embodiment, the saccharide cryoprotectant comprises a disaccharide. In a further preferred embodiment, the saccharide cryoprotectant comprises trehalose dihydrate.

According to an embodiment, the saccharide cryoprotectant may be present in the compositions in a concentration of between about 1 mM to about 50 mM, preferably between about 10 mM to about 30 mM, and still more preferably between about 15 mM to about 25 mM, inclusive of all integers within these ranges.

### Emulsifier

The compositions preferably include an emulsifying agent that can enhance the deposition of stored biological systems or materials onto a microanalysis device (e.g., a μPAD) and increase the capacity for resuspension of the stored biological matter in rehydration solution. The emulsifying agent may provide additional secondary benefits, such as functioning as a binding agent. Any suitable, non-toxic emulsifying agent may be used. In a preferred embodiment, the emulsifying agent comprises a cellulose or cellulose derivative.

Examples of suitable cellulose emulsifying agents include, without limitation, microcrystalline cellulose, powdered cellulose, cellulose, cellulose acetate, hydroxypropyl methylcellulose, carboxymethylcellulose, sodium carboxymethylcellulose, ethylcellulose, methylcellulose, hydroxyethylcellulose, hydroxyethylcellulose, hydroxypropylcellulose, or a combination thereof.

Other emulsifying agents acceptable for use in the compositions include, without limitation, acacia, alginic acid, gelatin, liquid glucose, guar gum, povidone, calcium carbonate, dibasic calcium phosphate, tribasic calcium phosphate, calcium sulfate, a dextrate, dextrin, dextrose, fructose, kaolin, lactose, mannitol, sorbitol, starch, pregelatinized starch, sucrose, diethanolamine, glyceryl monostearate, lecithin, monoethanolamine, oleic acid, oleyl alcohol, a poloxamer, polysorbate, sodium lauryl sulfate, stearic acid, sorbitan monolaurate, sorbitan monostearate, and other sorbitan derivatives, a polyoxyl derivative, wax, an ethylene oxide or propylene oxide emulsifier, or a combination thereof.

The emulsifier may be present in an amount of between about 0.1% to about 10%, preferably between about 0.2% to about 5%, and still more preferably between about 0.5% to about 3%, inclusive of all integers within these ranges.

### Lubricating Flow Agent

The compositions further preferably include one or more lubricating flow agents that reduce inter particular friction and improve the rate of powder/solid flow. In an embodiment, the compositions include two lubricating flow agents. Any suitable lubricating agent or flow agent may be used including, for example, a fatty acid salt, particularly an alkali metal or alkaline earth metal salt of a fatty acid, fatty acid, hydrocarbon, fatty alcohol, fatty acid ester (such as glycerol monostearate or butyl stearate), alkyl sulfate, polymers, or other suitable inorganic material.

Suitable fatty acids, including those used as part of a fatty acid salt, fatty alcohol, or fatty acid ester, may include a carboxylic acid derived from or contained in an animal or vegetable fat or oil, an unsaturated fatty acid, or a saturated fatty acid. Examples of suitable unsaturated fatty acids include oleic acid, butyric acid, lauric acid, palmitic acid, benzoic acid, fumaric acid, stearic acid, or a combination thereof. More preferably, the fatty acid comprises stearic acid or a salt thereof or fumaric acid, or a salt thereof. Salts of fatty acids may be formed with any metal salt. Preferably the metal is an alkali metal such as lithium, sodium, and potassium, an alkaline earth metal such as magnesium, calcium, or barium, or a transition metal such as lead, zinc, or cadmium. In a preferred embodiment, the lubricant flow agent comprises an ester of fumaric acid, a metal salt of stearic acid, or a combination thereof. In a further preferred embodiment, the lubricating flow agent comprises sodium stearyl fumarate, magnesium stearate, or a combination thereof. In a still further preferred embodiment, the lubricant flow agent is a combination of sodium stearyl fumarate and magnesium stearate.

Other suitable lubricating flow agents comprise hydrocarbons such as paraffin wax, amine wax, montan wax or a derivative thereof, a glyceryl ester such as glyceryl monostearate, a long-chain ester such as cetyl palmitate, glyceryl behenate, mineral oil, polyethylene glycol, talc, hydrogenated vegetable oil, sodium chloride, sodium lauryl sulfate, talc, or a combination thereof.

The one or more lubricating flow agents may be present in the compositions in an amount of between about 0.5% and about 20%, preferably between about 1% and about 15%, or still more preferably between about 1% and about 8%, inclusive of all integers within these ranges. In an embodiment, the composition comprises a first lubricating flow agent and a second lubricating flow agent. According to such an embodiment, the first lubricating flow agent, the second lubricating flow agent, or both lubricating flow agents may each be present in an amount of between about 0.1% to about 10%, preferably between about 1% and about 8%, or still more preferably between about 2% to about 6%, inclusive of all integers within these ranges.

### Additional Excipients

The compositions described herein can include one or more excipients in addition to other excipients, cryoprotectants, and lubricants described herein. Examples of suitable additional excipients include, for example, a high molecular weight polymer such as polyethylene glycol or a polyoxypropylene block polymer, DMSO, a carbon-based material, a cellulose-based material such as sodium carboxymethyl cellulose, microcrystalline cellulose (MCC), ethyl cellulose, cellulose acetate, hydroxypropylmethyl cellulose, and derivatives thereof, a metal or salt thereof or an inorganic salt such as alumina, aluminum stearate, magnesium stearate (Mg2S), or sodium stearyl fumarate (SSF), lecithin, a protein, phosphate, glycine, sorbic acid, potassium sorbate, polyacrylate, wax, lanolin, a sugar such as lactose, glucose, sucrose or trehalose dihydrate, a polysaccharide such as maltodextrin, pectin, guar gum, inulin, chondroitin sulfate, alginate, chitosan, or a cyclodextrin, a starch such as corn starch or potato starch, powdered tragacanth, malt, gelatin, glycerin, sorbitol, mannitol, talc, cocoa butter, an ester such as ethyl oleate or ethyl laurate, agar, a buffering agent such as magnesium hydroxide or aluminum hydroxide, alginic acid, ethyl alcohol, polyester, polycarbonate or a combination thereof.

More particularly, examples of a suitable carbon-based material include, without limitation, carbon, carbon black, graphite carbon, acetylene black, Ketjen Black, carbon nanotubes, carbon fibril, graphite, carbon fiber, or a combination thereof. In an embodiment, the compositions are free of carbon-based materials, such as graphite. In an embodiment, the compositions are free of DMSO.

### Additional Components

In addition to the aforementioned components, the assay compositions may further include one or more optional additional ingredients, such as a singlet oxygen quencher, a preservative/stabilizer, a buffering agent, an excipient, a bulking agent, a dispersing agent, a solubilizer, a solvent, a solidification agent, or any combination thereof.

A singlet oxygen quencher is a compound capable of reacting singlet oxygen often by virtue of reactive a electrons or lone pairs of sufficiently low ionizing energy. Singlet oxygen quenchers can in particular protect against the undesirable effects of oxygen. Examples of suitable singlet oxygen quenchers include, but are not limited to, alkyl imidazoles (e.g., histidine, L-carnosine, histamine, imidazole 4-acetic acid), indoles (e.g., tryptophan and derivatives thereof, such as N-acetyl-5-methoxytryptamine, N-acetylserotonin, 6-methoxy-1,2,3,4-tetrahydro-beta-carboline), sulfur-containing amino acids (e.g., methionine, ethionine, djenkolic acid, lanthionine, N-formyl methionine, felinine, S-allyl cysteine, L-selenocysteine, S-[2-(4-pyridyl)ethy]-L-cysteine, S-diphenylmethyl-L-cysteine, S-trityl-homocysteine, L-cysteine, N-acetyl-cysteine, S-ally-L-cysteine sulfoxide, S-aminoethyl-L-cysteine), phenolic compounds (e.g., tyrosine and derivatives thereof), aromatic acids (e.g., ascorbate, salicylic acid, and derivatives thereof), azides such as sodium azide, tocopherol, and related vitamin E derivatives, carotene or a carotenoid, and related vitamin A derivatives.

A preservative or stabilizer is any compound that inhibits the growth of microorganisms and that may be included in the compositions either before lyophilization or during resuspension/rehydration. Examples of suitable preservatives include, but are not limited to, sodium fluoride, citrate, dextrose, ACD, CPD, CPDA-1, sodium azide, a polyethylene glycol such as polyethylene glycol 200 (PEG 200), polyethylene glycol 300 (PEG 300), polyethylene glycol 400 (PEG 400), polyethylene glycol 540 (PEG 540), polyethylene glycol 600 (PEG 600), polyethylene glycol 1000 (PEG 1000), polyethylene glycol 1450 (PEG 1450), polyethylene glycol 3350 (PEG 3350), polyethylene glycol 4000 (PEG 4000), polyethylene glycol 4600 (PEG 4600), polyethylene glycol 8000 (PEG 8000), Carbowax MPEG 350, Carbowax MPEG 550, Carbowax MPEG 750, methyl paraben, ethyl paraben, propyl paraben and/or butyl paraben, sorbic acid, sodium sorbate, calcium sorbate, and/or potassium sorbate, benzoic acid, sodium benzoate, potassium benzoate, and/or calcium benzoate, sodium metabisulfite, propylene glycol, BHT, BHA, benzaldehyde, an essential oil, a phenol, or a combination thereof.
or a combination thereof. In some embodiments, the assay compositions include a preservative that preserves the activity of molecules that are dried (e.g., lyophilized), such as a sugar. Examples of suitable sugars include sucrose, fructose, trehalose, or a combination thereof.

Buffers may be used to modify or maintain the pH of the compositions either before lyophilization or during resuspension. Examples of suitable pH buffers include, without limitation, pH buffers include citric acid, tartaric acid, malic acid, sulfosalicylic acid, sulfoisophthalic acid, oxalic acid, borate, CAPS (3-(cyclohexylamino)-1-propanesulfonic acid), CAPSO (3-(cyclohexylamino)-2-hydroxy-1-propanesulfonic acid), EPPS (4-(2-hydroxyethyl)-1-piperazinepropanesulfonic acid), HEPES (4-(2-hydroxyethyl)piperazine-1-ethanesulfonic acid), MES (2-(N-morpholino)ethanesulfonic acid), MOPS (3-(N-morpholino)propanesulfonic acid), MOPSO (3-morpholino-2-hydroxypropanesulfonic acid), PIPES (1,4-piperazinediethanesulfonic acid), TAPS (N[tris(hydroxymethyl)methyl]-3-aminopropanesulfonic acid), TAPSO(2-hydroxy-3-[tris(hydroxymethyl)methylamino]-1-propanesulfonic acid), TES (N-[tris(hydroxymethyl)methyl]-2-aminoethanesulfonic acid), bicine (N,N-bis(2-hydroxyethyl)glycine), tricine (N-[tris(hydroxymethyl)methyl]glycine), tris (tris(hydroxymethyl)aminomethane), bis-tris(2-[bis(2-hydroxyethyl)amino]-2-(hydroxymethyl)-1,3-propanediol), 5-(4-dimethyl)amino benzylidene rhodamine, sulfosalicylic acid, lithium chloride, and lithium hydroxide, lithium dodecyl sulfate, or a combination thereof.

### Methods of Solid-State Storage

Methods of stabilizing a biological system may comprise contacting the biological system with a composition comprising a polymeric cryoprotectant, a saccharide cryoprotectant, an emulsifier, and a lubricating flow agent to form a mixture, lyophilizing the mixture, and forming the mixture into a solid. When it is time to use the biological system, the method further comprises a step of rehydrating the mixture. The method optionally further comprises a step of depositing the rehydrated mixture onto a substrate or analytical device, such as a paper-based microfluidic device.

### Composition Preparation

The compositions described herein may be prepared by combining a polymeric cryoprotectant, a saccharide cryoprotectant, an emulsifier, and a lubricating flow agent, together with a component in need of stabilizing, e.g., a biological system. In an embodiment, where the biological system is a CFPS platform, the biological system comprises cellular extract prepared from one or more solutions comprising between about 0.5 mL to 5 mL dNTPs, between about 10 μL to 100 μL of nicotinamide adenine dinucleotide (NAD), between about 10 μL to 200 μL Coenzyme A (CoA), between about 10 μL to about 100 μL oxalic acid, between about 20 μL to about 100 μL of putrescine, between about 50 μL to about 200 μL of spermidine, and between about 500 μL to about 1500 μL of 4-(2-hydroxyethyl)-1-piperazineethanesulfonic acid (HEPES) buffer. In an alternative embodiment, the cellular extract solution comprises between about 0.5mL to 5mL of 15× Salt Solution, between about 200 μL to about 1000 μL of 20 Amino Acid solution, and between about 200 μL to about 800 μL of Phosphoenolpyruvate (PEP).

### Lyophilization

After combining the composition with the biological system, the resulting mixture may then be lyophilized. In particular, the mixture may be flash frozen, e.g., by using liquid nitrogen, and thereafter placed into lyophilization beakers. Alternatively, lyophilization can occur using a large-scale or industrial freezer or freeze-dryer. In such an embodiment, the compositions as prepared may be of a volume acceptable for industrial-scale operations. The lyophilization may occur at any suitable temperature, for example between about 0° C. to about −80° C., preferably at about −50° C.

In an embodiment, the lyophilization time is between 1 hour and 24 hours, preferably between about 2 hours and about 3 hours, or between about 14 hours and about 24 hours. In an embodiment, where the volume of the composition being lyophilized is between about 10 μL and about 20 μL, preferably about 15 μL, the lyophilization time is between about 2 hours and bout 3 hours. In a further embodiment, where the volume of the composition being lyophilized is between about 500 μL and about 1000 μL, preferably 800 μL, the lyophilization time is between about 14 hours and about 24 hours.

### Solid Formation

The lyophilized mixture may subsequently undergo additional processing to form the mixture into a desired solid form. As used herein, the term “solid” refers to a solid such as a powder, flowable powder, a particle, an agglomerate, a flake, a granule, a pellet, a tablet, a pencil, or the like.

Prior to forming a solid, excipients may be introduced to the lyophilized mixture, and further mixed for desired distribution. In some embodiments, the excipients added at this step may be flow agents, lubricants, stabilizers, cryoprotectants, or a combination thereof.

For example, in order to form a solid pellet, the lyophilized mixture in the presence or absence of excipients may be transferred into a simple mortar and pestle in order to sufficiently crush the mixture into a powder. Other methods of crushing or mixing may be substituted. The resulting powder may then be deposited into a pellet press, such as a manual Parr Company pellet press (3.18 mm punch and die), or an automated pellet press, or equivalent device capable of pressing the powder into a solid form. The punch and die dimensions can range from 1 μm to 100 cm. The shape and profile of the solid may be formed in any geometric shape that is solid or hollow, such as a sphere, a cylinder, torus, prism, or cuboid. The dimensions and aspect ratios of the solid form may be adjusted to optimize surface-area-to-volume ratios. The surfaces may have additional shape features such as dimples and grooves. In other embodiments, the form factor may be a tablet. The tablet may be round, square, rectangle, capsule, almond, pentagon, oval, lozenge, diamond, triangle, core rod, and may have a profile that is shallow, standard, deep, or ball shape. Additional solidification aids, stabilizers, and binders may be added to-and mixed with the lyophilized material in order to ensure the lyophilized mixture remains in solid form.

Further discussion of the formation and characterization of reagent pencils is found, for example, in Liu et al., Characterization of Reagent Pencils for Deposition of Reagents onto Paper-Based Microfluidic Devices, Micromachines (Basel), 2017 August 5;8(8):242, which is herein incorporated by reference in its entirety. Similarly, discussion regarding the formation of lyophilized pellets is found, for example, in U.S. Application No. 2007/0259348, which is herein incorporated by reference in its entirety.

### Resuspension and Deposition

Methods of rehydrating or resuspending a lyophilized sample are disclosed herein. Preferably, the methods of rehydration or resuspension occur at a scaled-up volume, specifically a volume sufficient to permit the use of a solid, e.g., pellet platform. In an embodiment, the methods of rehydrating comprise combining the solid (e.g., a pellet) with a liquid, such as recommended media, broth, or water, to form a sample mixture in a container, wherein the reaction vessel volume is 0.01 mL-1,000 mL.

In some embodiments, the reaction vessel is incubated in a position to maximize the surface area of the sample mixture (e.g., incubating a test tube or centrifuge tube on its side), and refrigerating the sample mixture. In an embodiment, the refrigeration occurs at a temperature of between about 1° C. and about 10° C., preferably between about 3° C. to about 5° C., and still more preferably about 4° C. In a further embodiment, the refrigeration occurs for a period of between about 10 minutes to about 1 hour, preferably between about 20 minutes to about 40 minutes, still more preferably for about 30 minutes.

Alternatively, after solid formation, the solid may be deposited onto a substrate, such as paper-based device, and subsequently rehydrated. The rehydration solution may vary depending on the specific biological system stabilized in the solid. For example, the rehydration solution may comprise water, a solution comprising between about 0.5 mL to 5 mL dNTPs, between about 10 μL to 100 μL of nicotinamide adenine dinucleotide (NAD), between about 10 μL to 200 μL Coenzyme A (CoA), between about 10 μL to about 100 μL oxalic acid, between about 20 μL to about 100 μL of putrescine, between about 50 μL to about 200 μL of spermidine, and between about 500 μL to about 1500 μL of 4-(2-hydroxyethyl)-1-piperazineethanesulfonic acid (HEPES) buffer; or a solution comprising between about 0.5 mL to 5 mL of 15× Salt Solution, between about 200 μL to about 1000 μL of 20 Amino Acid solution, and between about 200 μL to about 800 μL of Phosphoenol pyruvate (PEP).

Where the solid is a reagent pencil or a pellet, the pencil or pellet can be used to deposit reagents on a device by simply drawing on the device to create a pencil trace. Alternatively, where the solid-state biological system has been resuspended and is a liquid, deposition on an analytical device may occur by preparing a solution of the reagent in an appropriate solvent, depositing a volume of the reagent solution on the device, and then drying the device to remove the solvent, thus leaving the dry reagent on the device. In a further embodiment, where the stabilized biological system has been resuspended and is in the form of a liquid, the liquid can be deposited onto a substrate having a non-porous barrier or well capable of holding the liquid for further use.

### Analytical Devices

In some embodiments, the solid-state storage system may be used in conjunction with one or more analytical devices, particularly one or more microscaled devices. In a preferred embodiment, the analytical device is a point of care testing device. The analytical device may further incorporate other features permitting complex functions, for example, the integration of nanostructures, electrodes, wax-printed butyrylcholinesterase (BChE) paper sensors, or surface functionalization through any suitable method, such as thin film deposition, plasma etching, lamination, or the like. Further discussion of surface functionalization of microfluidic devices can be found, for example, in Eichler, M., Klages, C. -P., & Lachmann, K. (2016). Surface Functionalization of Microfluidic Devices. Microsystems for Pharmatechnology, 59-97. doi: 10.1007/978-3-319-26920-7_3, which is herein incorporated by reference in its entirety. Suitable types of analytical devices include a microscale device, optical immunosensor, spectrometer, a benchtop analytical device such as a blood gas analyzer, infrared sensor, meter, microfluid analytical device, such as a microfluidic chip, or a microfluidic paper-based analytical device, test strip, or a combination thereof. One or more analytical devices may operate in tandem, e.g., a paper-based test strip comprising a biological component such as an enzyme and an electrode or other feature on a paper-based substrate that interfaces with a meter comprising a sensor and/or an electrode.

### Microfluidic Devices

The solid-state storage system may be used with or incorporated into one or more microfluidic devices such as microfluidic chips or paper-based microfluidic devices. Microfluidic chips comprise a pattern of microchannels engraved or etched into a material such as glass, silicon, paper, or a polymer like PDMS. The diameter of the microchannels typically ranges between about 100 nm and about 100 microns. This network of microchannels is connected to the macro-environment through one or more entry points on the chip. Fluid enters the chip through one or more flow control devices in order to achieve a desired effect, such as mixing, pumping, reacting, sorting, or controlling the biochemical environment. Additionally, low-cost paper-based microfluidic analytical devices are a useful type of lab-on-chip (LOC) platform that permits on and off-site analysis. In particular, microfluidic paper-based analytical devices (μPADs) provide rapid operation and precise interpretations while still being cost-effective. Another benefit is that μPADs are highly compact, portable, easy to use, and do not require additional sophisticated equipment to operate. Overall, microfluidic devices provide multi-process functionality compatible with a wide variety of biological systems. For example, the solid-state storage system may be used in conjunction with microfluidic PCR, qPCR, RT-PCR, qRT-PCR, pH control, drug administration, cell analysis, or diagnostics.

In a preferred embodiment, the analytical device comprises a paper-based microfluidic device. In some embodiments, the paper-based microfluidic device is used in conjunction with a storage chamber for use with samples that have been stored in a solid-state. Some traditional paper-based microfluidic devices can demonstrate incompatibility with samples that have been stored in a solid-state. To address this problem, in some embodiments, a resuspended sample deposited on a paper-based microfluidic device may further be stored in a chamber during incubation, wherein the chamber size is sufficient to prevent evaporation. In a further embodiment, water is added to the chamber in an amount sufficient to increase the vapor pressure in the chamber.

Alternatively, or additionally the paper-based microfluidic device may be laminated to improve compatibility with samples that have been stored in a solid-state. Excessive lamination of the paper-based microfluidic device, such as lamination of the entire channel or well, may inhibit the activity of the stored sample, potentially due to the lack of oxygen availability in the channel. Alternatively, the use of a minimal hydrophobic barrier may be insufficient to prevent leakage from the microfluidic device. Accordingly, in some embodiments, the entire microfluidic device is laminated except for the one or more wells or reaction chambers where the intended reaction would take place.

In some embodiments, the paper-based microfluidic device comprises a porous substrate preferably comprising paper, an input channel, a reaction chamber, one or more microfluidic channels, and a non-porous barrier that defines one or more boundaries for the reaction chamber and the one or more microfluidic channels, wherein the reaction chamber is fluidly connected to the input chamber by the one or more microfluidic channels, and wherein the surface of the microfluidic device is laminated except for the reaction chamber. In an embodiment, the non-porous barrier comprises wax. In an embodiment, the paper comprises nitrocellulose acetate, cellulose acetate, cellulosic paper, filter paper, tissue paper, writing paper, paper towel, cloth, or porous polymer film. The paper-based microfluidic device may comprise one or more input channels and one or more reaction chambers. Additionally, the surface of the microfluidic device, the reaction chamber, or the one or more channels may be functionalized. In some embodiments, the paper-based microfluidic device further comprises one or more conductive metals (e.g., Sn, Zn, Au, Ag, Ni, Pt. Pd, Al, In, or Cu), one or more polymers (e.g., a photoresist or a curable polymer), one or more insulating materials, or a combination thereof.

### Methods of Making Paper-Based Microfluidic Devices

In an embodiment, the analytical device comprises a paper-based microfluidic device with selective lamination to promote sample retention of resuspended samples. A method of making a paper-based microfluidic device generally comprises depositing a non-porous material onto a porous substrate, using the non-porous material to form one or more microchannels, a reaction chamber, an input, and optionally an output on the porous substrate, exposing the porous substrate and non-porous material to heat to form a paper-based microfluidic device, and laminating the surface of the paper-based microfluidic device except for the reaction chamber. The movement of fluid through the paper-based microfluidic device may be controlled by wicking, electric current, pump, micropump, syringe, or a combination thereof.

More particularly, a suitable paper-based microfluidic device may be designed using any suitable software, such as Adobe Illustrator or an open-source software tool such as AutoPAD, that permits design choices such as size and location of channels or wells and the addition of wax channels as layers on top of the paper base layer. Once designed according to the desired experimental specifications, the μPAD may be printed as a single sheet using, for example, a 3D printer, thermal transfer printer, or a wax printer. When a wax printer is used, the μPAD sheet is subsequently exposed to heat (e.g., in an oven) to support the formation of three-dimensional microfluidic wax channels.

In some embodiments, lamination is added to the μPAD for the purposes of either preventing evaporation of reaction solution off of the μPAD channels or preventing spillage of reaction solution off of the μPAD wells. The lamination substance includes any hydrophobic substance that can coat or integrate into the substrate. In some embodiments, the lamination substance could be a wax, a polymer, or a plastic. Unlaminated regions are maintained on the substrate as test areas to allow for solid deposition and introduction of rehydration solution. A laser cutter may be used to accurately cut circular holes in a sheet of the laminated layer that lined up with the desired test regions of the μPADs. After laser cutting, the laminate sheet is lined up against the μPAD sheet and laminated via a laminator. If more than one μPADs are printed on a single sheet, the μPAD sheets may subsequently be cut into individual μPADs.

The embodiments being thus described, it will be obvious that the same may be varied in many ways. Such variations are not to be regarded as a departure from the spirit and scope of the disclosure and all such modifications are intended to be included within the scope of the following claims.

### Rapid Prototyping. On-Demand Biomanufacturing, and Research Applications

The compositions and methods disclosed herein may be used to stabilize and store complex biological systems for a variety of applications. Examples of such applications include, without limitation, cell-free protein synthesis applications, CRISPR applications, biomanufacturing, research and development, screening, and prototyping. In some embodiments, the solid-state storage system may be deployed for applications involving aqueous phase reactions. In some embodiments, the solid pellet may be introduced to a rehydration solution in its entirety, or in smaller sections of the solid pellet to support target reaction volumes. Alternatively, in order to support larger reaction volumes, multiple solid pellets may be added, or a larger pellet may be used. The reactions may be run in droplet microfluidic devices, microtiter plates such as 384- and 96-well plates, tubes, or bioreactors. In some embodiments, the reactions may be run in batch mode, semi-continuous, or continuous exchange reactors.

The biomanufacturing process generally has at least four stages: bioproduction, recovery, purification, and bulk storage. “Bioproduction” refers to any way in which a biomolecule may be formed, modified, synthesized, or replicated. Following purification of a biomolecule, the manufacturing process includes formulation and storage steps. Formulation typically includes resuspending the biomolecule in a final solution containing physiologically acceptable excipients and carriers and/or lyophilizing the biomolecule formulation, aseptically processing the formulation, and finally, storing the biomolecule formulation in bulk. In the instant case, the solid-state storage systems disclosed herein may be utilized at any stage in the biomanufacturing process, for example by the stabilization and storage of the biological system needed to initiate bioproduction (e.g., reagents, catalysts, etc.) or product generated by the biomanufacturing process (e.g., the biomolecule).

Similarly, the solid-state storage systems and methods described herein may be used in prototyping applications, for example cell-free pathway prototyping. The development and engineering of biological systems for industrial applications often requires laborious, time-consuming, and costly design-build-test cycles. Cell-free systems offer rapid and simple approaches to studying cellular processes, largely through the study of single-enzyme activity assays and multienzymatic systems (both purified and crude). By decoupling cellular growth objectives from enzyme pathway engineering objectives, cell-free systems provide an inexpensive controllable environment to direct substrates toward a single, desired product. In prototyping applications, cell-free systems may be used to test and optimize biosynthetic pathways before implementation in live cells and scale-up. An example of cell-free synthetic biological methods that generalizable to any biosynthetic pathway of interest is found in Karim & Jewett, Cell-Free Synthetic Biology for Pathway Prototyping, Methods in Enzymology (2018) which is herein incorporated by reference in its entirety.

### Cell-Free Protein Synthesis (CFPS) Applications

Cell-free protein synthesis platforms are one type of biological system that may be stabilized in a solid state according to the compositions and methods disclosed herein. Cell-free protein synthesis platforms use crude cell extracts to produce target proteins, including bioactive recombinant DNA (rDNA) proteins. CFPS platforms generally require (1) a genetic template (mRNA or DNA) encoding the target protein and (2) a reaction solution containing the necessary transcriptional and translational components (e.g., cell extracts), such as RNA polymerases for mRNA transcription, ribosomes for polypeptide translation, tRNA and amino acids, enzymatic cofactors and an energy source(s), and/or cellular components essential for proper protein folding. Cell extracts may be made from any suitable source, such as E. coli, rabbit reticulocyte lysate (RRL), wheat germ, insects, or humans.

Any part or all of a CFPS platform may be stabilized according to the present disclosure. More particularly, CFPS platforms can be used to prepare a sequence-defined biopolymer of protein in vitro. The platforms for preparing a sequence-defined polymer or protein in vitro comprise a cellular extract from an organism and in particular a strain of E. coli, such as BL21*DE3. Because CFPS exploits an ensemble of catalytic proteins prepared from the crude lysate of cells, the cellular extract (whose composition is sensitive to growth media, lysis method, and processing conditions) is an important component of extract-based CFPS reactions. A variety of methods exist for preparing an extract competent for cell-free protein synthesis, including those disclosed in U.S. Application No. 2014/0295492, which is herein incorporated by reference. The platform may comprise an expression template, a translation template, or both an expression template and a translation template. The expression template serves as a substrate for transcribing at least one RNA that can be translated into a sequence-defined biopolymer (e.g., a polypeptide or protein). The translation template is an RNA product that can be used by ribosomes to synthesize the sequence-defined biopolymer. In certain embodiments, the platform comprises both the expression template and the translation template. In certain specific embodiments, the platform may be a coupled transcription/translation (“Tx/Tl”) system where the synthesis of a translation template and a sequence-defined biopolymer are from the same cellular extract.

The CFPS platform may also comprise one or more polymerases capable of generating a translation template from an expression template comprised of a circular plasmid, linear DNA template, or an RNA template. The polymerase may be supplied exogenously or may be supplied from the organism used to prepare the extract. In certain specific embodiments, the polymerase is expressed from a plasmid present in the organism used to prepare the extract and/or an integration site in the genome of the organism used to prepare the extract.

The CFPS platform may also comprise one or more chaparone capable of facilitating protein folding during or after in vitro translation. The chaparone may be supplied exogenously or may be supplied from the organism used to prepare the extract.

The platform may comprise an orthogonal translation system. An orthogonal translation system may comprise one or more orthogonal components that are designed to operate parallel to and/or independently of the organism's orthogonal translation machinery. In certain embodiments, the orthogonal translation system and/or orthogonal components are configured to incorporate amino acids. An orthogonal component may be an orthogonal protein or an orthogonal RNA. In certain embodiments, an orthogonal protein may be an orthogonal synthetase. In certain embodiments, the orthogonal RNA may be an orthogonal tRNA or an orthogonal rRNA. An example of an orthogonal rRNA component has been described in U.S. Application Nos. 2017/0073381 and 2016/0060301, which are herein incorporated by reference in their entirety. In certain embodiments, one or more orthogonal components may be prepared in vivo or in vitro by the expression of an oligonucleotide template. The one or more orthogonal components may be expressed from a plasmid present in the genomically recoded organism, expressed from an integration site in the genome of the genetically recoded organism, co-expressed from both a plasmid present in the genomically recoded organism and an integration site in the genome of the genetically recoded organism, expressed in the in vitro transcription and translation reaction, or added exogenously as a factor (e.g., an orthogonal tRNA or an orthogonal synthetase added to the platform or a reaction mixture).

The CFPS platform to be stabilized may include a platform used for preparing a sequence-defined biopolymer or protein in vitro, where the platform comprises a cellular extract prepared from a cell culture of a species of E. coli. In particular, the strain of E. coli may include the strain BL21*DE3, based on the Cell-Free AutoInduction (CFAI) method. The CFPS platform may also include purified and reconstituted in vitro transcription translation systems, or cell extracts from other prokaryotic or eukaryotic organisms. DH5alpha cells may be utilized as the plasmid propagation strain for DNA purification. Plasmids used may comprise, for example, a pJL1 plasmid such as pJL1-sfGFP or a pY71 plasmid, such as pY71-mRFP1. According to such a platform, BL21*DE3 cells may be cultured. As the BL21*DE3 cells used for extract preparation have no antibiotic resistance cassette, no antibiotic is required in the culture. Following incubation, the culture may be induced with Isopropyl β-D-1-thiogalactopyranoside (IPTG) and further combined with a buffer comprising the following reagents: 1.4M Mg(OAc)2, 1M, 8.2 pH Tris(OAc), and 6M KOAc, along with 200 μL 1M Dithiothreitol (DTT) in 96.8 mL of water. Serial centrifugation and vortexing result in the formation of cell pellets. The cell pellets may be flash frozen using liquid nitrogen, resuspended, and sonicated. After sonication, the resuspended cell pellets have undergone lysis of their cell membranes to form cell lysate. After lysis, 4.5 μL of IM DTT is added to each tube and mixed via inversion.

The cellular extract of the platform is prepared from one or more solutions containing molecular grade ultrapure H₂O, cell extract, and DNA, solutions containing energy, salts, and other cofactors. More particularly, the cellular extract solution may comprise a 1 mL master mix of dNTPs, 60 μL of Nicotinamide adenine dinucleotide (NAD), 80μL of Coenzyme A (CoA), 60 μL of Oxalic acid, 60 μL of Putrescine, 90 μL of Spermidine, and 855 μL of 4-(2-hydroxyethyl)-1-piperazineethanesulfonic acid (HEPES) buffer. In an alternative embodiment, the cellular extract solution may comprise 1 ml of 15× Salt Solution, 600 μL of 20 Amino Acid solution, and 494 μL of Phosphoenolpyruvate (PEP). In another embodiment, the buffer conditions may be oxidizing to promote disulfide bonds.

More generally, the CFPS platform to be stabilized may include one or more additional biological components, for example, one or more components for performing CFPS. Components may include, but are not limited to amino acids which optionally may include noncanonical amino acids, NTPs, salts (e.g., sodium salts, potassium salts, and/or magnesium salts), cofactors (e.g., nicotinamide adenine dinucleotide (NAD) and/or coenzyme-A (CoA)), an energy source and optionally an energy source comprising a phosphate group (e.g., phosphoenol pyruvate (PEP), ATP, or creatine phosphate), a translation template (e.g., a non-native mRNA that is translated in the platform) and/or a transcription template (e.g., a template DNA for synthesizing a non-native mRNA that is translated in the platform), and any combination thereof.

In some embodiments, the platform may comprise an energy source and optionally an energy source comprising a phosphate group (e.g., phosphoenol pyruvate (PEP), ATP, or creatine phosphate), where the energy source is present in the platform at a concentration of greater than about 30 mM, 40 mM, 50 mM, 60 mM, 70 mM, 80 mM, or 90 mM (preferably greater than about 67 mM), but less than about 100 mM, or within a concentration range bounded by of these values.

In some embodiments, the platform further comprises a source of potassium, such as a potassium salt such as potassium glutamate, where the platform comprises potassium at a concentration of between about 50 mM to about 500 mM, inclusive of all integers in this range, such as 100 mM, 150 mM, 200 mM, 250 mM, 300 mM, 350 mM, 400 mM, or 450 mM.

Further description of strains and cell-free protein synthesis methods that may be stabilized according to the present disclosure may be found, for example, in, U.S. Pat. Nos. 5,478,30; 5,556,769; 5,665,563; 6,168,931; 6,869,774; 6,994,986; 7,118,883; 7,189,528; 7,338,789; 7,387,884; 7,399,610; 8,703,471; and 8,999,668, along with U.S. Application Nos. 2015/0259757, 2014/0295492, 2014/0255987, 2014/0045267, 2012/0171720, 2008/0138857, 2007/0154983, 2005/0054044, and 2004/0209321, 2005/0170452; 2006/0211085; 2006/0234345; 2006/0252672; 2006/0257399; 2006/0286637; 2007/0026485; 2007/0178551; and 2018/0016612, which are herein incorporated by reference in their entirety. Additionally, suitable methods, procedures, and materials for storage for cell-free expression systems are found in Brookwell A W, Gonzalez J L, Martinez A W, Oza J P. Development of Solid-State Storage for Cell-Free Expression Systems. ACS Synth. Biol. 2023 September 15;12(9):2561-2577. doi: 10.1021/acssynbio.3c00111, which is herein incorporated by reference in its entirety.

### Clustered Regularly Interspersed Short Palindromic Repeats (CRISPR) Applications

Genome editing systems represent one type of biological system that may be stabilized according to the compositions and methods disclosed herein. The CRISPR-Cas9 system functions by utilizing a short, noncoding guide RNA (gRNA) that has at least two molecular components: a target-specific CRISPR RNA (crRNA) and an auxiliary trans-activating CRISPR RNA (tracrRNA), although the system can also include a CRISPR enzyme complexed with the guide RNA. The gRNA guides the Cas9 protein to a specific genomic locus via base pairing with the target sequence. Upon binding to the target sequence, the Cas9 protein induces a specific double-strand break. Following DNA cleavage, the break is repaired through nonhomologous end joining (NHEJ) or homology-directed repair (HDR) mechanisms. There are several mechanisms available for CRISPR-Cas9 delivery. For example, in the DNA delivery format, the CRISPR DNA vector enters the cell and translocates to the nucleus, where the Cas9 mRNA and gRNA are transcribed. The Cas9 protein, as translated in the cytoplasm, combines with the gRNA to form a ribonucleoprotein (RNP) complex that then enters the nucleus for targeted gene editing. In the RNA delivery format, the Cas9 mRNA and gRNA are cotransfected into the cell cytoplasm, where the mRNA is translated to produce functional Cas9 protein. The Cas9-gRNA (RNP) protein delivery format streamlines cell engineering by eliminating transcription and translation in the cell. With the RNP format, there is no requirement for a specific promoter as the Cas9 RNP complex can act immediately after it enters the cell since transcription and translation are not required.

Particularly relevant systems are those comprising molecules capable of introducing a double-strand break (“DSB”) or single-strand break (“SSB”) at a specific site or sequence in a double-stranded DNA, such as in genomic DNA or in a target gene located within the genomic DNA as well as accompanying guide RNA or donor or other DNA template polynucleotides. Examples of such gene editing molecules include: (a) a nuclease comprising an RNA-guided nuclease, an RNA-guided DNA endonuclease or RNA directed DNA endonuclease (RdDe), a class 1 CRISPR type nuclease system, a type II Cas nuclease, a Cas9, a SpCas9, a nCas9 nickase, a type V Cas nuclease, a Cas12a nuclease, a nCas12a nickase, a Cas12d (CasY), a Cas12e (CasX), a Cas12b (C2c1), a Cas12c (C2c3), a Cas12i, a Cas12j, a Cas12L, a Cas14, an engineered nuclease, a codon-optimized nuclease, a zinc-finger nuclease (ZFN) or nickase, a transcription activator-like effector nuclease (TAL-effector nuclease or TALEN) or nickase (TALE-nickase), an Argonaute, and a meganuclease or engineered meganuclease; (b) a polynucleotide encoding one or more nucleases capable of effectuating site-specific alteration (including introduction of a DSB or SSB) of a target nucleotide sequence; (c) a guide RNA (gRNA) for use with an RNA-guided nuclease, or a DNA encoding a gRNA for use with an RNA-guided nuclease; (d) donor DNA template polynucleotides suitable for insertion at a break in genomic DNA by homology-directed repair (HDR) or microhomology-mediated end joining (MMEJ); and (e) other DNA templates (e.g., dsDNA, ssDNA, or combinations thereof) suitable for insertion at a break in genomic DNA (e.g., by non-homologous end joining (NHEJ).

In certain embodiments, an RNA-guided endonuclease that leaves a blunt end following cleavage of the target site is part of the biological system. Blunt-end cutting RNA-guided endonucleases include Cas9, SpCas9, Cas12c, Cas12i, and Cas12h. In certain embodiments, an RNA-guided endonuclease that leaves a staggered single-stranded DNA overhanging end following cleavage of the target site is part of the biological system. Staggered-end cutting RNA-guided endonucleases include Cas12a, Cas12b, and Cas12e.

Other CRISPR elements may be included in the biological systems that are stored according to the methods and compositions, e.g., gene editing molecules comprising CRISPR endonucleases and CRISPR guide RNAs including single guide RNAs or guide RNAs in combination with tracrRNAs or scoutRNA, or polynucleotides encoding the same. In certain embodiments, the biological system comprises CRISPR elements provided in the form of isolated molecules, as isolated or semi-purified products of a cell-free synthetic process (e.g., in vitro translation), or as isolated or semi-purified products of a cell-based synthetic process (e.g., such as in bacterial or other cell lysates). In certain embodiments, the CRISPR elements can comprise a transgene that expresses a CRISPR endonuclease (e.g., a Cas9, a Cpf1-type, or other CRISPR endonucleases). In certain embodiments, one or more CRISPR endonucleases with unique PAM recognition sites can be present in the biological system.

The biological system can also comprise guide RNAs (sgRNAs or crRNAs and a tracrRNA) that form an RNA-guided endonuclease/guide RNA complex that binds sequences in the gDNA target site that are adjacent to a protospacer adjacent motif (PAM) sequence. The type of RNA-guided endonuclease typically informs the location of suitable PAM sites and the design of crRNAs or sgRNAs. G-rich PAM sites, e.g., 5′-NGG are typically targeted for the design of crRNAs or sgRNAs used with Cas9 proteins. Examples of PAM sequences include 5′-NGG (Streptococcus pyogenes), 5′-NNAGAA (Streptococcus thermophilus CRISPR1), 5′-NGGNG (Streptococcus thermophilus CRISPR3), 5′-NNGRRT or 5′-NNGRR (Staphylococcus aureus Cas9, SaCas9), and 5′-NNNGATT (Neisseria meningitidis). Cas9 variants from Streptococcus thermophilus (StCas9), Neisseria meningitidis (NmCas9), Francisella novicida (FnCas9), and Campylobacter jejuni (CjCas9) may also be used.

T-rich PAM sites (e.g., 5′-TTN or 5′-TTTV, where “V” is A, C, or G) are typically targeted for the design of crRNAs or sgRNAs used with Cas12a proteins. In some instances, Cas12a can also recognize a 5′-CTA PAM motif. Other examples of potential Cas12a PAM sequences include TTN, CTN, TCN, CCN, TTTN, TCTN, TTCN, CTTN, ATTN, TCCN, TTGN, GTTN, CCCN, CCTN, TTAN, TCGN, CTCN, ACTN, GCTN, TCAN, GCCN, and CCGN (wherein N is defined as any nucleotide). Cpf1 endonuclease and corresponding guide RNAs and PAM sites are disclosed in US Patent Application No. 2016/0208243, which is incorporated herein by reference for its disclosure of DNA encoding Cpf1 endonucleases and guide RNAs and PAM sites.

More specifically, for the purposes of gene editing, the biological system can comprise CRISPR arrays that have been designed to contain one or multiple guide RNA sequences corresponding to a desired target DNA sequence. Such guide RNA sequences typically have a length of 17-24 nucleotides and have a high degree of complementarity to the targeted gene.

The biological systems may also comprise other nucleases capable of effecting site-specific modification of a target nucleotide sequence, such as zinc-finger nucleases (ZFNs), transcription activator-like effector nucleases (TAL-effector nucleases or TALENs), Argonaute proteins, a meganuclease or an engineered meganuclease. Zinc finger nucleases (ZFNs) are engineered proteins comprising a zinc finger DNA-binding domain fused to a nucleic acid cleavage domain, e.g., a nuclease. The zinc finger binding domains provide specificity and can be engineered to specifically recognize any desired target DNA sequence. The zinc finger DNA binding domains are derived from the DNA-binding domain of a large class of eukaryotic transcription factors called zinc finger proteins (ZFPs). The DNA-binding domain of ZFPs typically contains a tandem array of at least three zinc “fingers” each recognizing a specific triplet of DNA.

Transcription activator-like effectors (TALEs) are proteins secreted by certain Xanthomonas species to modulate gene expression and to facilitate the colonization by and survival of the bacterium. Studies of TALEs have revealed the code linking the repetitive region of TALEs with their target DNA-binding sites. TALEs comprise a highly conserved and repetitive region consisting of tandem repeats of mostly 33 or 34 amino acid segments. A strong correlation between unique pairs of amino acids at a particular position(s) and the corresponding nucleotide in the TALE-binding site allows for the design of DNA binding domains of any desired specificity. TALEs can be linked to a non-specific DNA cleavage domain to prepare genome-editing proteins, referred to as TAL-effector nucleases or TALENs. As in the case of ZFNs, a restriction endonuclease, such as Fokl, can be conveniently used and therefore be part of the biological systems.

Argonautes are proteins that can function as sequence-specific endonucleases by binding a polynucleotide (e.g., a single-stranded DNA or single-stranded RNA) that includes sequence complementary to a target nucleotide sequence) that guides the Argonaut to the target nucleotide sequence and effects site-specific alteration of the target nucleotide sequence.

In related embodiments, the biological systems further comprise other functional domains used in conjunction with zinc finger nucleases, TALENs, or Argonautes. Examples of functional domains include transposase domains, integrase domains, recombinase domains, resolvase domains, invertase domains, protease domains, DNA methyltransferase domains, DNA hydroxyl methylase domains, DNA demethylase domains, histone acetylase domains, histone deacetylase domains, nuclease domains, repressor domains, activator domains, nuclear-localization signal domains, transcription-regulatory protein (or transcription complex recruiting) domains, cellular uptake activity associated domains, nucleic acid binding domains, antibody presentation domains, histone modifying enzymes, recruiter of histone modifying enzymes; inhibitor of histone modifying enzymes, histone methyltransferases, histone demethylases, histone kinases, histone phosphatases, histone ribosylases, histone deribosylases, histone ubiquitinases, histone deubiquitinases, histone biotinases, and histone tail proteases. Non-limiting examples of functional domains include a transcriptional activation domain, a transcription repression domain, and an SHH1, SUVH2, or SUVH9 polypeptide capable of reducing the expression of a target nucleotide sequence via epigenetic modification.

In some embodiments, the CRISPR enzyme is part of a fusion protein comprising one or more heterologous protein domains (e.g., about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity, and nucleic acid binding activity, Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRPU), chlioramphenicol acetyltransferase (CAT) beta-galactosidase, beta-glucuronidase, luciferase,
green fluorescent protein (GFP), HcRed, DsRed. cyan fluorescent protein (CFP), yellow fluorescent protein (YFP), and auto-fluorescent proteins including blue fluorescent protein (BFP). A CRISPR enzyme may be fused to a gene sequence encoding a protein or a fragment of a protein that binds DNA molecules or bind other cellular molecules, including but not limited to maltose-binding protein (MBP), S-tag, Lex A DNA binding domain(DBD) fusions, GAL4 DNA binding domain fusions, and herpes simplex virus (HSV) BP16 protein fusions.

In some embodiments, a tagged CRISPR enzyme is used to identify the location of a target sequence. In an aspect of the invention, a reporter gene which includes but is not limited to glutathione-S-transferase (iST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CUP), yellow fluorescent protein (YFP), and autofluorescent proteins including blue fluorescent protein (BFP), may be introduced into a cell to encode a gene product which serves as a marker by which to measure the alteration or modification of expression of the gene product. In a further embodiment of the invention, the DNA molecule encoding the gene product may be introduced into the cell via a vector.

In some embodiments, the biological systems further comprise one or more polynucleotides or vectors that drive the expression of one or more polynucleotides and/or genome editing systems. In certain embodiments, a polynucleotide vector comprises a regulatory element such as a promoter operably linked to one or more polynucleotides encoding the polypeptide or genome editing system, such as constitutive, conditional, inducible, and temporally or spatially specific promoters (e.g., a tissue-specific promoter, a developmentally regulated promoter, or a cell cycle-regulated promoter). Examples of developmentally regulated promoters include Phospholipid Transfer Protein (PLTP), fructose-1,6-bisphosphatase protein, NAD(P)-binding Rossmann-Fold protein, adipocyte plasma membrane-associated protein-like protein, Rieske [2Fe—2S] iron-sulfur domain protein, chlororespiratory reduction 6 protein, D-glycerate 3-kinase, chloroplastic-like protein, chlorophyll a-b binding protein 7, chloroplastic-like protein, ultraviolet-B-repressible protein, Soul heme-binding family protein, Photosystem I reaction center subunit psi-N protein, and short-chain dehydrogenase/reductase proteins.

Expression vectors or polynucleotides that are part of the biological systems may also contain a DNA segment near the 3′ end of an expression cassette that acts as a signal to terminate transcription and directs polyadenylation of the resultant mRNA and may also support promoter activity. Such a 3′ element is commonly referred to as a “3′-untranslated region” or “3′-UTR” or a “polyadenylation signal.”

In certain embodiments, the biological systems further comprise a vector or polynucleotide comprising an expression cassette that includes additional components, e.g., a polynucleotide encoding a drug resistance or a polynucleotide encoding a detectable marker such as green fluorescent protein (GFP) or beta-glucuronidase (gus) to allow convenient screening or selection of cells expressing the vector or polynucleotide. More particularly, the biological system may comprise an mRFP1 gRNA expressing gRNA specific to mRFP1 (RFP) expression. Further discussion of CRISPR-Cas systems and methods can be found in U.S. Pat. No. 8,697,359, which is herein incorporated by reference in its entirety.

### Applications for Other Biological Systems

The solid-state storage systems described herein are capable of being adapted to stabilize and store a wide variety of biological systems. In addition to CFPS and CRISPR applications, the compositions and methods of the present disclosure can be incorporated into a diagnostic system, for example, a biological system for identifying pathogens or diagnosing disorders having a genetic marker. For example, the storage systems described herein can be used as part of rapid prototyping, directed evolution, and biomanufacturing applications, including at a large or industrial scale. The storage systems and kits may also be used for identifying heavy metals and/or small molecule contaminants.

More particularly, the reagents required for a diagnostic test may be provided as part of a diagnostic kit comprising the compositions described herein and a μPAD or comparable microfluidic device. The microfluidic device is preferably integrated with nanostructures, electrodes, and/or sensors or has a functionalized surface to enable diagnostic testing. The kit optionally further comprises one or more additional analytical devices, such as another microscale device, optical immunosensor, spectrometer, a benchtop analytical device such as a blood gas analyzer, infrared sensor, meter, or the like. The kit may optionally comprise means for collecting a biological sample.

The kit may be prepared according to a method comprising (i) combining a composition comprising a polymeric cryoprotectant, a saccharide cryoprotectant, an emulsifier, and a lubricating flow agent, with a component in need of stabilizing to form a mixture; (ii) lyophilizing the mixture; (iii) forming the mixture into a solid stabilized component. The kit may be used according to a further step comprising (iv) contacting the solid stabilized component with a liquid to form a resuspension solution. Following resuspension, the component may be used for its intended use. Alternatively, following resuspension, the method may comprise an additional step of (v) depositing the resuspension solution onto or in an analytical device, such as a μPAD. At this point, the resuspension solution may be used for its intended purpose (e.g., diagnostics, testing, research, reaction, etc.). Optionally, the method may further comprise (vi) collecting a biological sample; (vii) reacting the resuspension solution with the biological sample to generate a result, and (viii) interpreting the result.

The biological sample used in conjunction with the methods and compositions disclosed herein may be derived from a biological fluid including, but not limited to blood, saliva, semen, urine, amniotic fluid, cerebrospinal fluid, synovial fluid, vitreous fluid, gastric fluid, nasopharyngeal aspirate lymph fluid, or a combination thereof. The biological sample can also be a tissue sample, a water sample, an air sample, a food sample, or a crop sample.

The biological sample used in conjunction with the methods and compositions disclosed herein may be derived from recombinant expression, or chemically or enzymatically synthesized.

The compositions, methods, and kits described herein can be used to diagnose and detect a wide variety of heavy metals, small molecule contaminants, pathogens, disorders, markers, flags, or biomarkers. For example, the compositions and methods described herein can be used to provide point-of-care or cold chain-free diagnostic testing in the fields of bacterial or viral infections, oncology, medical genetics, emergency medicine, immunology and allergies, prenatal screening, identity testing, education, research, screening and the like. Further discussion of such diagnostic applications can be found, for example, in Jung et al., Cell-Free Biosensors for Rapid Detection of Water Contaminants, NAT. BIOTECH., 2020 38:1451-1459, Jung et al., Programming Cell-Free Biosensors with DNA Strand Displacement Circuits, NAT. CHEM. BIO., 2022 18:385-393, which are both herein incorporated by reference in their entirety.

The compositions and methods disclosed herein can be employed within instrumentation capable of converting any biological or chemical material into a solid state. The instrumentation may be industrial or commercial. Such an instrument may integrate a lyophilizer with a mixer and a press to provide plug-and-play production of solid-state materials.

The compositions and methods disclosed herein can be employed in one or more of the following areas, including point-of-incidence and real-time pathogen detection or clinical diagnostics. Further uses include the genotyping of an organism, for purposes of determining the incidence of, or predisposition to, genetic diseases or incorporation into a drug monitoring device. In another embodiment, the compositions and methods can be employed in the area of industrial and agricultural monitoring. For example, the present disclosure can be used in conjunction with systems for monitoring and/or detecting metals, organic molecules, toxins, pathogens born by food, crops, livestock, and the like. In another embodiment, the present disclosure can be used in conjunction with forensics tests. For example, the present invention can be used to genetically identify an individual.

In one embodiment, genetic disorders and disorders having a genetic component can be diagnosed by employing the system and method of the present invention. For example, numerous oncogenes, tumor markers, and other biomarkers have been identified and implicated in the development of breast, colorectal and other cancers. Such markers and genes (including mutations thereof) include, for example, p53, c-erbB2, ALK, Alpha-fetoprotein (AF), B-cell immunoglobulin, BCL2, Beta-2-microglobulin (B2M), beta-human chorionic gonadotropin (Beta-hCG), bladder tumor antigen (BTA), BRCA1, BRCA2, BCR-ABL, BRAF V600, C-kit-CD117, CA15-3/CA27.29, CA19-9, CA-125, CA 27.29, calcitonin, carcinoembryonic antigen (CEA), CD19, CD20, CD22, CD25, CD30, CD33, chromogranin A (CgA), chromosome 17p deletion, chromosomes 3, 7, 17, and 9p21 (for use in monitoring bladder cancer recurrence), circulating tumor cells of epithelial origin (CELLSEARCH), cytokeratin fragment 21-1, cyclin D1 (CCND1), des-gamma-carboxy prothrombin (DCP), DPD, EGFR, estrogen receptor (ER), progesterone receptor (PR), FGFR2, FGFR3, fibrin/fibrinogen, FLT3, gastrin, HE4, HER2/neu, 5-HIAA, IDH1 and IDH2, Immunoglobulins, IRF4. JAK2, KRAS. Lactate dehydrogenase, MYC. microsatellite instability (MSI) (e.g., in connection with colorectal cancer), MYD88, Myeloperoxidase (MPO), Neuron-specific enolase (NSE), NTRK, Nuclear matrix protein 22, PCA3 mRNA, PML/RARα, Prostatic Acid Phosphatase (PAP), Programmed death ligand 1 (PD-L1), Prostate-specific antigen (PSA), ROS1, Soluble mesothelin-related peptides (SMRP), Somatostatin receptor, T-cell receptor, Terminal transferase (TdT), Thiopurine S-methyltransferase (TPMT), Thyroglobulin, UGT1A1*28, Urine catecholamines (VMA and HVA), Urokinase plasminogen activator (uPA), plasminogen activator inhibitor (PAI-1), FoundationOne CDx (F1CDx), Guardant360 CDx, 5-Protein signature (OVA1), 17-Gene signature, 21-Gene signature, 46-Gene signature, 70-Gene signature, and the like.

Infectious agents which can be diagnosed using the compositions and methods of the present disclosure include, but are not limited to, bacteria, viruses, fungi, actinomycetes, and parasites. Examples include, but are not limited to, Escherichia. Shigella, Salmonella, Arizona (Salmonella subgenus III), Citrobacter, Klebsiella, Enterogacter, Serratia, Proteus, Providentia, Morganella; Vibrio and Campylobacter; Brucella such as undulant fever; Yersinia; Pasteurella; Francisella; Actinocacillosis; Haemophilus, Bordetella (e.g. Burdetella pertussis); Pseudomonas and Legionella; Bacteroides, Fusobacterium, Streptobacillus and Calymmatobacterium; Bacillus (spore forming aerobes); Clostridium(spore-forming anaerobes); Lissteria and Erysipelothrix; Corynebacterium; Mycobacterium; Spirochetes; Rickettsias; Chlamydia; Mycoplasmas; Poxviruses; Herpesviruses; Papovaviruses; Adenoviruses; Orthomyxoviruses, Paramyxoviruses; Rhabdoviruses; Cytomegalovirus; Retroviruses; Picornaviruses; Cornaviruses; Rotaviruses; Hepatitis viruses (e.g., hepatitis C virus, hepatitis B virus); Togaviruses; Bunyaviruses; Arenaviruses; Cryptococcus; Candida (e.g., Candida albicans, Candida glabrata, Candida parapsilosis, Candida tropicalis); Sporothrux; Ilestoplasma; Coccidioides; Blastomyces; Aspergilli; Zygomycetes; Dematiaceae; Fusarium; Protozoa; Nemathelminthes; Platyhelminthes; SARS-COV-2 variants and sub-variants such as the alpha, beta, gamma, and omicron variants and the BA.1, BA.2, BA.3, BA.4, BA.5, BA.2.12.1 and BA.2.75 sub-variants; and viruses from the Orthopoxvirus, such as Human monkeypox (MPX).

In other embodiments, retroviruses such as HIV-1, HIV-2, any of HIV-1 Groups M, N, O, or P, any of HIV-1 Group M subtypes A-K, or any other known type or subtype of HIV, HTLV-1 and HTLV-2 and herpesviruses such as HSV-1, HSV-2, VZV, EBV, CMV, and HHV-6 can be detected. In still other embodiments, the disorders, and diseases that can be detected by use of systems incorporating the presently disclosed methods and compositions include, for example, Cystic fibrosis, Gaucher disease, Medium chain acyl dehydrogenase deficiency, Myotonic dystrophy, Sodium channelopathies, Chloride channelopathies, Duchenne/Becker muscular dystrophy.

### EXAMPLES

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

General Methods

Aseptic techniques were practiced for all experimental methods performed. Benchtops were regularly cleaned with 70% ethanol after use. For experiments requiring the use of RNAse, a special RNAse benchtop was utilized, and all instruments used were afterward cleaned with RNAse Away and 70% ethanol. Water was purified to nanopure specifications and used for reagent preparation, for CFPS reactions molecular grade ultrapure water was instead used.

Antibiotics included kanamycin and ampicillin, which were used in LB plate preparation and bacterial cell culture. For LB plate preparation LB agar was suspended in nanopure water and autoclaved on Liquid 30 setting, then cooled to 55° C. before deposition into Petri dishes and left overnight. For LB plates designated for bacterial cell growths with specific antibiotic resistances, the antibiotic of choice (1 mL antibiotic/1 L LB agar) was added just before deposition in the Petri dishes. Plates streaked with bacterial cells were incubated at 37° C. overnight and then stored at 4° C. for continuous use. All DNA samples were kept at 20° C. while all cell pellets, cell extract, glycerol stocks, and CFPS reagents were flash frozen using liquid nitrogen and stored at −80° C. All other chemicals and reagent components were stored according to their specific MSDS storage requirements.

### Bacterial Cell Strain Preparation

All bacterial strains utilized were variants of Escherichia coli optimized for expression or plasmid propagation. BL21*DE3 cells were utilized as the expression strain for extract preparation used for all CFPS reactions. DH5-alpha cells were utilized as the plasmid propagation strain for DNA purification. Both strains were utilized for DNA purification of CRISPR-Cas genetic constructs.

### Cell Extract Preparation

Cell extracts were prepared using the E. coli strain BL21*DE3, based on the Cell-Free Auto Induction (CFAI) method. BL21*DE3 cells were streaked on LB agar plates and incubated at 37° C. overnight, and a single colony was taken from the plate to inoculate 1 L of LB broth (Miller) in a 2 L TunAir Flask. The LB broth comprised 10 g/L NaCl, 10 g/L Tryptone, and 5 g/L of Yeast Extract, where 25 g of LB broth powder was suspended in 1 L of nanopure H₂O via magnetic stir bar mixing and autoclaved to remove any possible contaminants before inoculation. As the BL21*DE3 cells used for extract preparation have no antibiotic resistance cassette, no antibiotic was required in the 1 L culture. The 1L culture was then incubated in a shaking incubator at 37° C. and 200 rpm for about 14 hours before optical density (OD) measurements were taken. To measure OD, a spectrophotometer was set to wavelengths of 490-620 nm, clean cuvettes were prepared, and the instrument was calibrated with an LB broth blank of 0 at 600 nm. Once an OD of 0.6 was reached, the 1 L culture was induced with 1 mM of Isopropyl β-D-1-thiogalactopyranoside (IPTG) and from there the OD of the 1 L culture was measured every 30 minutes until an OD of 2.5 was reached. At this point, incubation was halted by taking the TunAir flask from the incubator and placing it in an ice bath which resulted in a final OD of ˜3.0 by the time of centrifugation. Before centrifugation, 100 ml of S30 buffer was prepared by combining 1 mL of the following reagents: 1.4M Mg(OAc)2, 1M, 8.2 pH Tris(OAc), and 6M KOAc, along with 200 μL 1M Dithiothreitol (DTT) in 96.8mL of nanopure H₂O.

To begin centrifugation, the IL culture was transferred from the TunAir flask to a cold 1 L centrifuge tube. All centrifugation steps were completed using a Beckman Coulter Avanti J-E centrifuge. After adding water to a second 1 L centrifuge and balancing the two tubes on a scale, the tubes were centrifuged for 10 minutes at 5000×g at 10 C in the JLA-9.1 rotor. After spinning the supernatant was discarded and a sterile spatula was used to transfer the cell pellet from the IL centrifuge tube to a cold 50 mL falcon tube. 30 ml of S30 buffer was then added to the falcon tube and the tube was pulse vortexed to resuspend the cell pellet. After adding water to a second 50 mL falcon tube and balancing the two tubes on a scale, the tubes were centrifuged for 10 minutes at 5000×g at 10 C in the J.S-5.3 rotor. After discarding the supernatant another 25 mL of S30 buffer was added to the tube, which was pulse vortexed and centrifuged again using the same parameters. After again discarding the supernatant, 30 mL of S30 buffer was added to the tube and the tube was pulse vortexed to resuspend the cell pellet. Following this, 10 mL of the resuspended cell pellet was transferred to 3 new 50 mL falcon tubes and balanced with a fourth 50 mL falcon tube containing water, then the 4 tubes were centrifuged for 10 minutes at 5000×g at 10° C. in the J.S-5.3 rotor. After discarding the supernatant, the three tubes containing the cell pellets were weighed, flash frozen using liquid nitrogen, then stored at −80C for further use in later extract preparation steps.

The next step of extract preparation involved the sonication of the resuspended cell pellets that were previously stored at −80° C. using a Qsonica Q125 sonicator with a 3.175 mm diameter probe. After S30 buffer was prepared using the same protocol outlined previously, 1 mL of S30 buffer per 1 g of cell pellet mass was added to the cell pellets. The pellets were then allowed to thaw on ice for between 30 and 60 minutes before they were resuspended via pulse vortexing. After resuspension, 1.4 mL of resuspended cell pellet was pipetted in 1.5 mL Eppendorf tubes and the tubes were placed in an ice water bath. The tubes containing the resuspended cell pellet were then sonicated for 3 bursts of 45 seconds ON, and 59 seconds OFF at an amplitude of 50%. After sonication, the resuspended cell pellets have undergone lysis of their cell membranes and were now referred to as cell lysate. After lysis, 4.5 μL of IM DTT was added to each tube and mixed via inversion. The tubes were then centrifuged for 10 minutes at 12,000×g at 4° C. After spinning the supernatant was transferred to new Eppendorf tubes which were flash frozen and stored at −80C for later use. To assess the quality of the cell extract, a CFPS reaction of pJL 1-sfGFP was performed using the new extract compared to a control extract that had already been determined to be of good quality. Once quantification of sfGFP fluorescence from the CFPS reaction confirms the quality of the new extract the new lot can be used for further experimentation.

### CFPS Reagent Preparation

Along with molecular grade ultrapure H₂O, cell extract, and DNA, solutions containing energy, salts, and other cofactors necessary for transcription and translation must be prepared to perform CFPS reactions. These solutions, dubbed “solution A” and “solution B”, contain the ingredients necessary to support transcription/translation reactions outside of the cell. Solution A was prepared in 2.205 mL batches, enough to support 1000 15 μL CFPS reactions. Solution A consists of a 1 mL master mix of dNTPs, 60 μL of Nicotinamide adenine dinucleotide (NAD), 80 μL of Coenzyme A (CoA), 60 μL of Oxalic acid, 60 μL of Putrescine, 90 μL of Spermidine, and 855 μL of 4-(2-hydroxyethyl)-1-piperazineethanesulfonic acid (HEPES) buffer. Solution B was prepared in 2.095 mL batches, enough to support 1000 15 μL CFPS reactions. Solution B consists of 1 mL of 15× Salt Solution, 600 μL of 20 Amino Acid solution, and 494 μL of Phosphoenolpyruvate (PEP).

### DNA Transformation & Purification

DNA constructs purified for this research include pJL1-sfGFP, pY71-mRFP1, pJL1-SpCas9, and pJL1-mRFP1 gRNA. DNA constructs pJL1-sfGFP pJL1-SpCas9 and pJL1-mRFP1 gRNA were purified from DH5alpha cells using an Invitrogen PureLink HiPure Plasmid Maxiprep Kit, where the constructs were eluted using incubated molecular grade ultrapure H2O and stored at −20° C. DNA construct pY71-mRFP1 was purified from BL21*DE3 cells using an Invitrogen PureLink HiPure Plasmid Miniprep Kit and transformed into chemically competent NEB5alpha cells following the New England BioLabs High Efficiency Transformation Protocol. After transformation, both BL21*DE3 and NEB5alpha cells containing the pY71-mRFP1 DNA construct were purified in parallel using an Invitrogen PureLink HiPure Plasmid Maxiprep Kit, eluted using incubated molecular grade ultrapure H2O and stored at −20C. All DNA constructs purified were validated for quality in CFPS reactions.

### Cell-Free Protein Synthesis

Regardless of volume, CFPS reactions consisted of roughly 33.33% molecular grade ultrapure water, 33.33% cell extract, 14.67% Solution A, 14.00% Solution B, and varying amounts of DNA depending on concentration and constructs involved. For lyophilized solutions, 80% of the total original volume was used for rehydration. All CFPS reactions were incubated at 37° C. Because the CFPS reactions expressed fluorescent proteins that originate from aquatic organisms that naturally exist at colder temperatures, after incubation the samples were allowed to cool at 4° C. for 30 minutes to improve fluorescence via more complete post-translational folding of the expressed proteins.

### Quantification & Data Analysis

Quantification of fluorescence of CFPS expressed proteins was achieved via the use of a Cytation 5 microplate reader and black-bottomed 96-well plates. For all wells, 2 uL of sample solutions was pipetted in 48 uL of HEPES buffer, with each condition being tested in triplicate resulting in 12 data points per condition. For sfGFP fluorescence, the plate reader program was set to an excitation wavelength of 485 nm and an emission wavelength of 510 nm, while for mRFP the plate reader program was set to an excitation wavelength of 584 nm and an emission wavelength of 607 nm. A previously validated standard curve for sfGFP was used to convert relative fluorescence units (RFU) into sfGFP concentration (ug/mL), while mRFP1 quantification was kept in RFU as no validated standard curve was available. Raw fluorescence data were analyzed in Microsoft Excel and quantitative results were correlated with qualitative visual observations made in visible light, HEV light, and UV imaging performed using a GelDoc imager.

### Example 1. Lyophilization Protocol

Development and validation of the lyophilization protocol for 15 μL CFPS reactions expressing pJL1-sfGFP were conducted.

### Lyophilization

Cell-free solutions lyophilized included 15 μL to 800 μL volume reactions containing molecular grade ultrapure H₂O, cell extract, solution A, solution B, and DNA in varying concentrations depending on total solution volume, DNA stock concentration, and excipient concentration. 15 uL-100 μL reactions were prepared and lyophilized in 1.5 mL Eppendorf tubes while 100 uL-800 μL reactions were prepared and lyophilized in 15 mL centrifuge tubes. All cell-free mixtures contained the cryoprotectant excipients 0.4% PEG8000 and 20 mM Trehalose Dihydrate previously demonstrated to improve the stability of cell-free reagents during lyophilization. After cell-free solutions were prepared, the tubes were flash frozen in liquid nitrogen and immediately placed into lyophilization beakers and attached to the lyophilizer. The lyophilizer maintained a sample temperature of ˜−52° C. and a vacuum of ˜0.280 mBar, where samples were lyophilized overnight. Lyophilized sample material not immediately used in pellet fabrication or other experimentation was stored at −20° C. with a desiccant.

### Validation

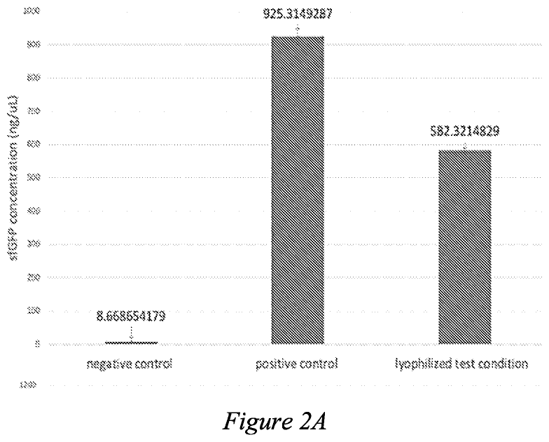

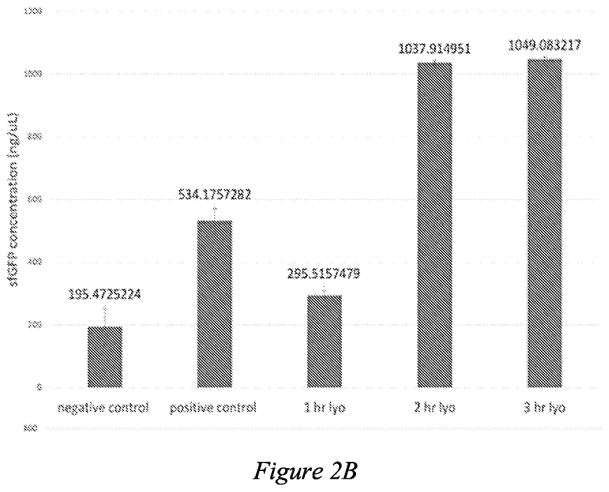

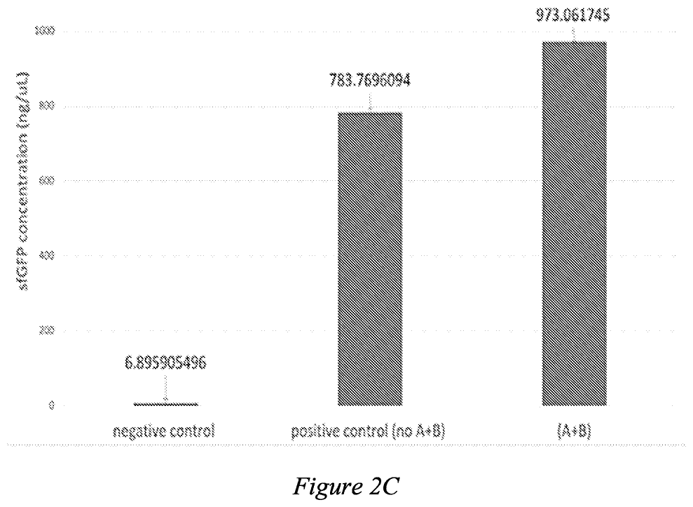

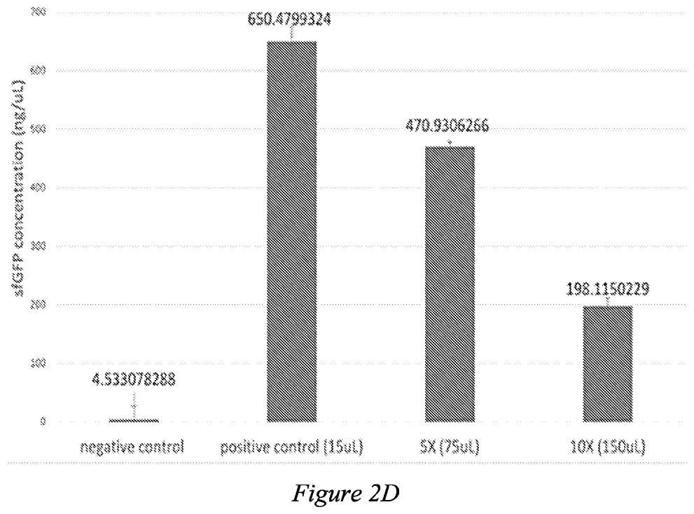

Molecular grade ultrapure water, cell extract, and pJL1-sfGFP DNA were all combined and flash frozen in liquid nitrogen and lyophilized. After lyophilization, the sample was rehydrated with 80% of the initial reaction volume and incubated at 37° C. along with aqueous controls. As shown in FIG. 2A, positive expression of pJL1-sfGFP was observed in the lyophilized trials but expression was decreased in comparison to aqueous positive controls.

To address this problem, the lyophilization time was modified. Additional experiments were performed evaluating 15 uL CFPS reactions at 1 hr, 2 hr, and 3 hr lyophilization time points. As shown in FIG. 2B, the 2 hr and 3 hr time points both showed greatly increased expression over the 1 hr lyophilization time point. Evaluating these results, a 3 hr lyophilization time was selected for the planned scaling of CFPS reactions. It was observed in later lyophilization time experiments that longer lyophilization times were detrimental to CFPS activity at smaller (e.g. 15 uL) volumes but beneficial to much larger (e.g., 1.125 mL) reactions. Accordingly, the selected lyophilization time for 800 uL reactions used for pellet fabrication was ˜20 hrs-24hrs.

To further improve lyophilization, energy and cofactor reagents used in CFPS reactions were added to the lyophilization mixture. The expression of these reactions was compared to ones where the lyophilized material was instead rehydrated with a master mix of molecular grade ultrapure water and these reagents. As shown in FIG. 2C, integration of the energy and cofactor reagents into the lyophilization mixture increased the expression over the previous mixture by ˜20%.

### Example 2. Scaled Reaction Volume

The successful scaling up of cell-free reactions has long been considered an industry need as part of more effectively transitioning cell-free platforms to industrial applications. Such scaling up has been validated in a variety of formats for several synthesized targets, although the development of a scaled, universal platform remains a need. with the baseline volume for validated expression of pJL1-sfGFP in CFPS reactions was 15 μL, the next step was to scale the CFPS reactions from this volume to observe differences in expression. A lyophilized CFPS experiment expressing pJL1-sfGFP at 15 μL (1×), 75 μL (5×), and 150 μL (10×) was initially run to assess how these reactions would be affected by scaling. As shown in FIG. 2D, scaling the CFPS reactions to 75 μL maintained >70% of pJL1-sfGFP expression while scaling to 150 μL resulted in a ˜70% decrease in expression. Without being bound by theory, it is thought that this decrease in expression at 150 μL could be due to factors such as the fragility of reaction components to lyophilization, limited oxygen availability upon rehydration, and limited surface area for the reaction to take place, all of which are known barriers to scaling.

These issues were first addressed by identifying cryoprotective excipients that could be integrated into the CFPS mixtures at concentrations where their cryopreserving properties would be useful without becoming toxic to the reactions. As described in further detail herein, it was found that a combination of 0.4% Polyethylene Glycol (PEG) 8000 and 20 mM Trehalose Dihydrate was beneficial at preserving CFPS activity in lyophilized samples.

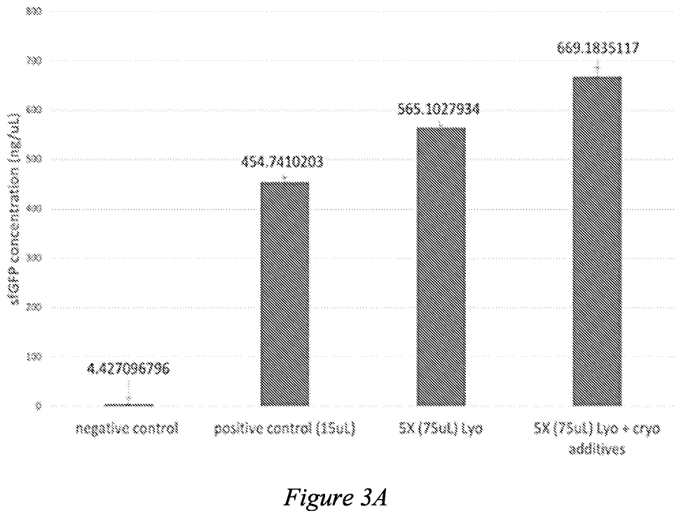

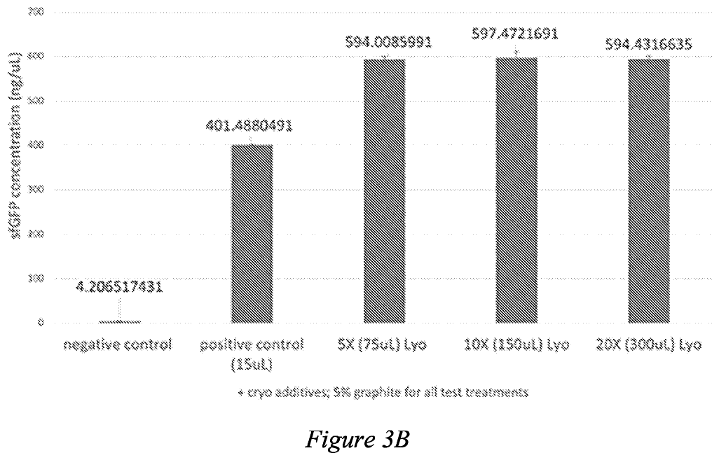

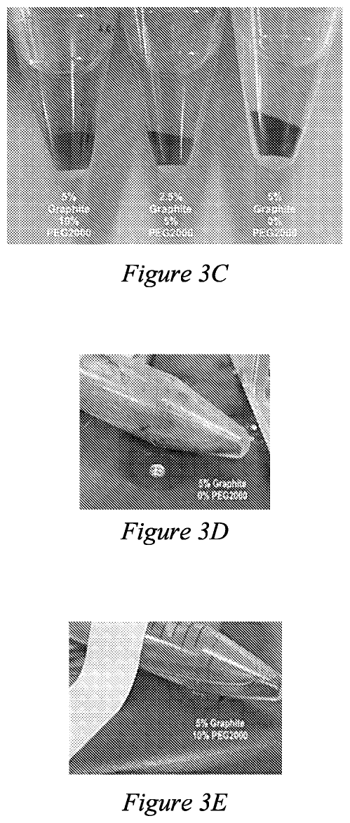

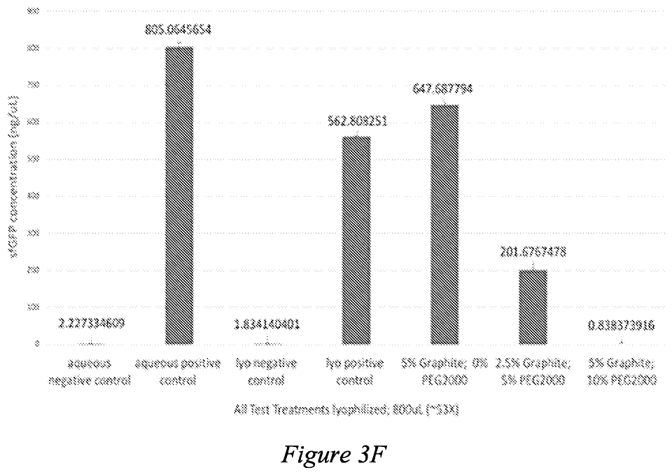

As shown in FIG. 3A, the addition of these cryoprotective excipients not only recovered but enhanced, CFPS activity beyond the lyophilized positive controls even when paired with the addition of excipients observed to be detrimental to expression. Further, as shown in FIG. 3B, the CFPS reactions showed no decrease in pJL1-sfGFP expression up to 300 μL (20X) reactions.

For the largest CFPS reactions tested at 800 μL (˜53×) and 1.125 ml (75 ×), the cryoprotective excipients were able to partially recover CFPS activity, however, a decrease compared to the controls was still observed. Consequently, consistent with FIG. 3C, FIG. 3D, FIG. 3E, and FIG. 3F, additional techniques were developed to increase pJL1-sfGFP expression at these larger volumes, such as replacing the previously used 1.5 mL Eppendorf tubes with 15 mL centrifuge tubes for the reactions to increase the oxygen available for use in CFPS. An additional useful step included incubating the 15 mL centrifuge tubes containing the reactions on their sides to increase the surface area of the CFPS reactions. Lastly, for all volumes of reactions tested, there was an additional step of refrigerating the CFPS reactions expressing pJL1-sfGFP for 30 minutes at 4° C. after incubation. The rationale is that Green Fluorescent Protein (GFP), is derived from the jellyfish Aequorea victoria, and so colder temperatures may increase post-translational folding. The addition of these techniques as well as the cryoprotective excipients was observed to sufficiently recover expression of pJL1-sfGFP in these scaled volumes, validating that the CFPS reactions could be scaled to the volumes necessary to be successfully utilized in a solid-stated (e.g., pellet) platform.

### Example 3. Identification of Excipients for Solid-State Storage

The addition of excipients has been utilized for the development of previously described reagent pencils to both stabilize the dyes or enzymes integrated into the pencil “lead” pellet and to enhance the “pencil-like” qualities of the pellets. For the integration of CFPS into a pellet platform, the rationale for integrating these excipients was to increase the pellets' capacity for resuspension in rehydration solutions as well as for deposition of pellet material onto μPADs. For these reasons the excipients had to first be validated in CFPS reactions so that the correct concentrations of these excipients could be identified that would modulate the material dynamics of the pellets to the desired specifications without detrimental loss of expression.

Multiple excipients were tested for their ability to improve the stability of reaction components during lyophilization and storage, increase capacity for resuspension of the pellets, increase capacity for deposition of the pellet material onto μPADs, and identify superior material factors for ease-of-use. Cryoprotectant excipients PEG8000 (polyethylene glycol) and trehalose dihydrate were prepared as a master mix for easy addition into cell-free reactions at concentrations of 0.4% and 20 mM, respectively. As described in greater detail herein, five main excipients for testing the other aforementioned metrics of pellets were evaluated: graphite, PEG2000, microcrystalline cellulose (MCC), sodium stearyl fumarate (SSF), and magnesium stearate (Mg2S). PEG2000 and MCC were added at varying concentrations during cell-free reaction preparation before lyophilization. SSF and Mg2S were also added at varying concentrations during the pressing of lyophilized material into pellets after lyophilization was completed. Combinations of these excipients as treatments in cell-free pellet experiments were also performed using the same procedure as the prior single excipient additions.

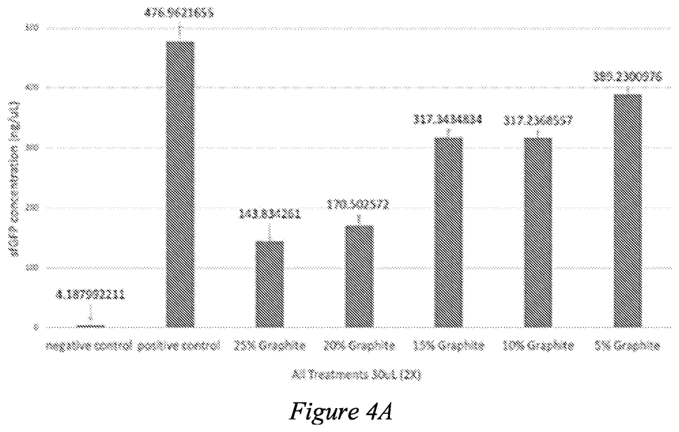

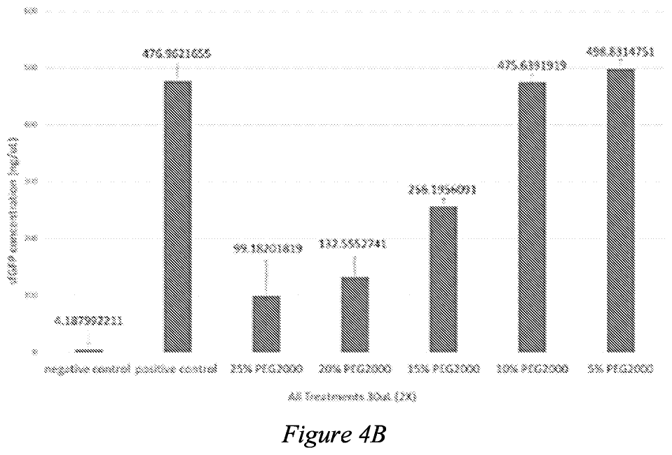

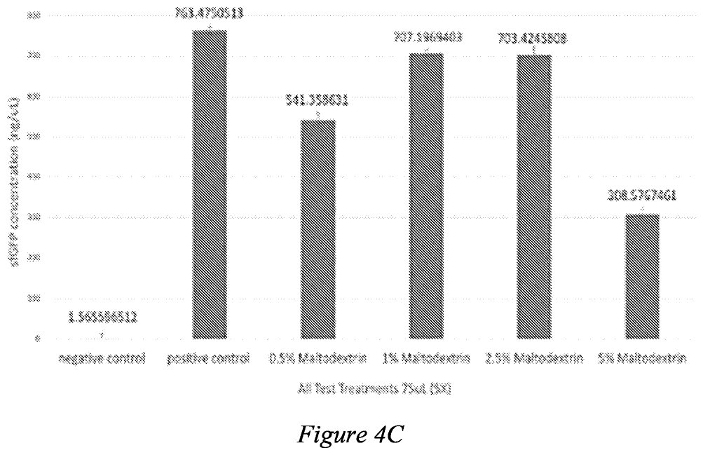

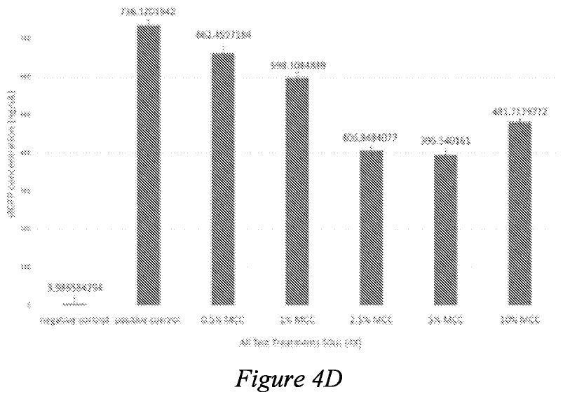

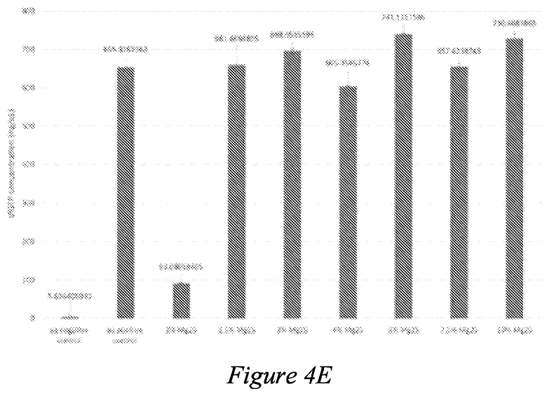

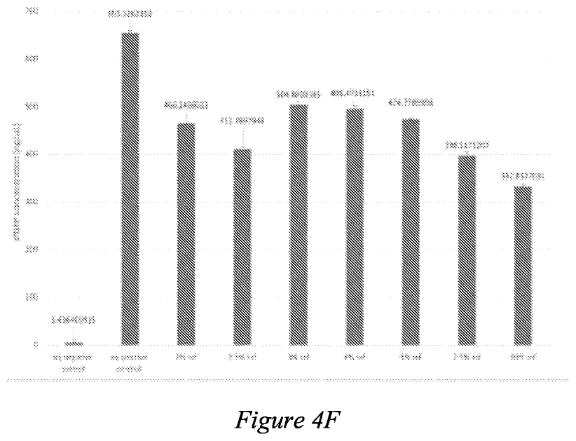

More particularly, the first excipients evaluated were PEG2000 and graphite because of their use in reagent pencils. Identification of successful concentrations of excipients began with excipient titration experiments, observing CFPS activity from 0% (positive control) to 25% graphite or PEG2000, increasing concentration in 5% increments. The results are shown in FIGS. 4A-4F. In these figures, pJL1-sfGFP expression decreased with the increase in the concentration of either excipient. In the graphite titration, the 5% treatment maintained >80% pJL1-sfGFP expression compared to the positive control, with a decrease in expression observed in the 10% and 15% trials, both displaying ˜66% of the expression observed in the positive control. Expression decreased sharply in the 20% and 25% trials, with both displaying <50% of the expression of the positive control. In the PEG2000 titration, expression was not affected in either the 5% or 10% trials, with each showing expression above and at the level of the positive control. A sharp decrease in expression was observed in the 15% to 25% trials, displaying a drop off in expression from ˜54% to ˜21% of the expression observed in the positive control. The addition of the cryoprotective excipients in replicate titration experiments improved expression in all treatments but the decreasing trend in expression with an increase in either PEG2000 or graphite concentration remained the same.

Evaluating the data collected from these experiments, it was determined that the preferred concentrations of graphite and PEG2000 were 5% and 10%, respectively, as these concentrations were observed to be the least detrimental to pJL1-sfGFP expression. Scaling experiments integrating either PEG2000 or graphite with cryoprotective excipients were successful in maintaining high pJL1-sfGFP expression, however when in combination with each other the two excipients were found to be detrimental to expression. As shown in FIG. 3F and FIG. 4A, this effect was observed when evaluating the combination of 5% graphite and 10% PEG2000 with 5% graphite alone for an 800 μL lyophilized reaction—the reaction volume for integration of CFPS into a pellet platform.

Expression in the 5% graphite, 800 uL trial was observed to be ˜14% higher than that of the 15 uL lyophilized positive control, likely due to the strategies for recovering expression in scaled-up, lyophilized reactions detailed previously. In comparison, the 5% graphite, 10% PEG2000, 800 uL trial was observed to display no pJL1-sfGFP expression. An additional 2.5% graphite, 5% PEG2000, 800 uL trial was observed to display greatly decreased expression at ˜35% of the lyophilized positive control. Taken cumulatively, these results demonstrated that the combination of graphite and PEG2000 was detrimental to the expression of pJL1-sfGFP and was therefore less preferable for integration into the pellet platform.

Next, further excipients were identified and validated for their effect on CFPS activity. These new excipients included maltodextrin, microcrystalline cellulose, sodium stearyl fumarate, and magnesium stearate. Maltodextrin, a polysaccharide commonly used in foo in d production, was identified as a lyoprotectant and tested in CFPS. To ascertain the effect maltodextrin has on CFPS activity, a maltodextrin titration from 0% (positive control) to 5% was performed in 75 uL CFPS reactions. The results are shown in FIG. 4C. Expression of pJL1-sfGFP remained stable with a nominal decrease in expression of <10% at up to 2.5% maltodextrin. Expression was observed to decrease sharply at 5% maltodextrin, displaying ˜40% of the expression of the positive control.

As the previously described cryoprotectant excipient formulation was observed to perform well in recovering CFPS activity, maltodextrin was deemed unnecessary for integration into the platform and was not pursued in further experiments. Microcrystalline cellulose (MCC) is a cellulose polymer utilized in cosmetic and pharmaceutical applications as an emulsifying agent. The rationale for the integration of MCC into a pellet platform was its capacity to enhance the deposition of pellet material onto μPADs, as well as increase the capacity for resuspension of pellets in rehydration solution. To ascertain the effect MCC has on CFPS activity, an MCC titration from 0% (positive control) to 10% was performed. Expression was observed to decrease ˜10% at 0.5% MCC, with a sharp decrease in expression at 2.5% to 10% MCC, maintaining <70% of the expression of the positive control. Evaluating these results, it was determined a concentration of 1% MCC would be utilized for further expression experiments. Sodium Stearyl Fumarate (SSF) and Magnesium Stearate (Mg2S) are lubricating flow agents utilized in applications such as food production and pharmaceuticals.

The rationale for the two excipients' integration into a pellet platform is two-fold. One, in their ability to act as flow agents streamlining the pellet manufacturing process, and two, in their capacity to break up the intermolecular interactions of the CFPS components to allow the pellet to more readily resuspend in rehydration solutions. Titration experiments of both excipients for 0% (positive control) to 10% in CFPS reactions were tested to ascertain their individual effects on CFPS activity. As shown in FIG. 4E, Mg2S titration displayed a general increasing trend in expression with an increase in Mg2S concentration, with an increase in expression to ˜11% above that of the positive control in the 10% treatment. Further, as shown in FIG. 4F, the SSF titration displayed a general trend of slightly decreasing pJL1-sfGFP expression with an increase in SSF expression, with a decrease in expression from ˜77% to ˜50% of the positive control expression from 3% to 10% SSF. As neither excipient was observed to be prohibitively detrimental to CFPS activity at higher concentrations, the optimal concentrations of each excipient were determined by pellet resuspension tests detailed in the next section.

### Example 4. Pellet Fabrication, Resuspension, and Deposition

Following the evaluation of Example 3, a protocol for pellet fabrication and excipient addition was developed. After the lyophilization protocol of Example 1, lyophilized samples were used as material for pellet fabrication. The lyophilized sample volume used for pellet fabrication was standardized at 800 uL as it was ascertained that this volume ensured enough lyophilized material for successful pellet fabrication. Tubes containing lyophilized material were weighed with an analytical balance so that the mass of the lyophilized material could be calculated by subtracting this value from the previously recorded mass of the empty tubes. Following this, lyophilized material was removed from the tube and deposited into a simple mortar and pestle where the material was sufficiently crushed into a powder and deposited into the pellet press. A manual Parr Company pellet press was used for all experiments involving pellet fabrication, with the 3.18 mm punch and die used to fabricate all pellets because of the lower mass requirement compared to the larger 6.35 mm punch and die. Pellets not immediately used in experimentation were stored at −20° C. with a desiccant.

After pellet fabrication, pellets were used either in experiments involving resuspension in Eppendorf tubes or deposition of pellet material onto μPADs followed by subsequent rehydration. Depending on the composition of the pellets involved, the rehydration solution either consisted of purely molecular grade ultrapure water or a master mix of solutions A, B, and molecular grade ultrapure water. Depending on the experiment, rehydration solution volume was either standardized at a set volume or calculated on a per pellet basis. This calculation involved taking the mass of the pellet and dividing it by the total mass of lyophilized material the pellet was fabricated from. This value was then multiplied by 80% of the original total volume of the solution before lyophilization, which was standardized at 640 uL for 800 uL reactions. In cases where pellets were segmented via scalpel, these individual segments were weighed using an analytical balance and the same procedure was followed. Following this partial resuspension, pellets were pulse vortexed until full resuspension could be achieved. The time the pellets or pellet segments were pulse vortexed was dependent on the excipients incorporated into the pellets or pellet segments, and this data was recorded to indicate the best excipients or excipient combinations for resuspension. To deposit lyophilized material onto μPADs, pellets were inserted into [pencil lead holders] and deposited in the same manner as the previously described reagent pencils. The success of replicating these pencil-like qualities varied depending on the excipients incorporated, and data on the best excipients for this was recorded. μPADs were weighed before and after the deposition of pellet material to ascertain the mass of the pellet material dispensed on the μPAD. For rehydration of pellet material onto the μPADs, 15 uL of rehydration solution was standardized and pellet mass deposited on μPADs was controlled as much as possible to remove possible error from the results.

### Example 5. Prototyping Fabrication, pJL1-sfGFP Expression & Excipient Additions After Solid-State Storage

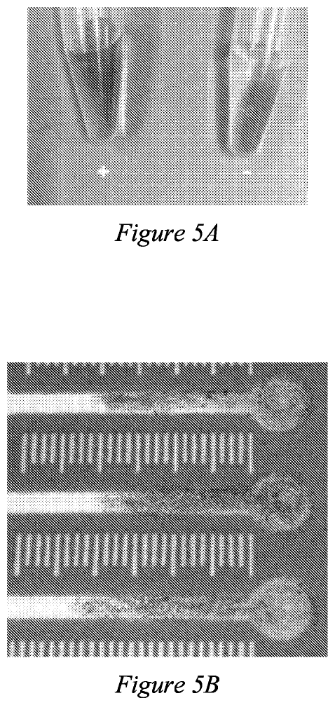

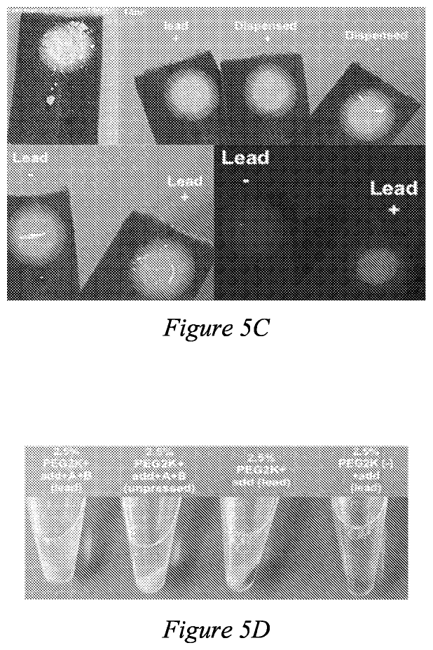

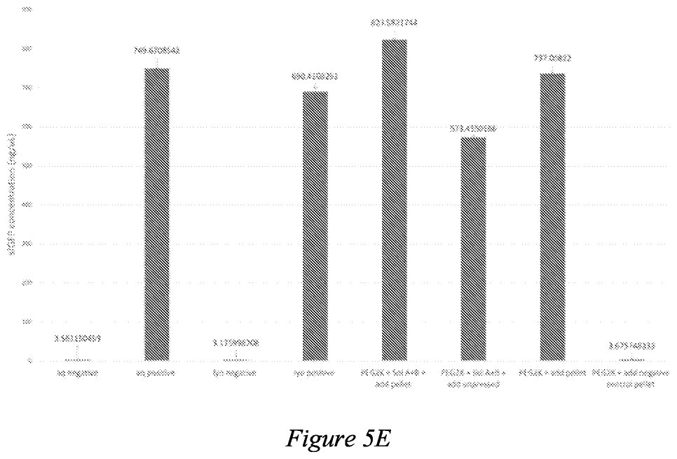

Following the lyophilization, scaling, and excipient factors validated in CFPS reactions, the next step was to validate the fabrication of CFPS pellets and their expression of pJL1-sfGFP. Pellets were prepared according to the protocols of the prior examples, comprising horseradish peroxidase (HRP) and Glucose Oxidase (GOx) enzymes. As shown in FIG. 5A and FIG. 5B, the activity of the reagent pencils was evaluated through the use of ABTS liquid reagent. where enzymatic activity was confirmed colorimetrically through both resuspension of the test pellet as well as deposition of the pellet material through writing on μPADs. From this experiment, the ability to fabricate pellets that support biochemical reactions was validated. Through evaluation of the fabrication process, the fabrication of 3.18 mm diameter pellets was standardized, as these were found to be less reagent-costly than larger diameter pellets.

Initial attempts fabricating CFPS pellets for expression of pJL1-sfGFP followed the scaled-up formulation, an 800 μL CFPS reaction utilizing molecular grade ultrapure water, cell extract, and pJL1-sfGFP DNA with 0.4% PEG8000, 20 mM trehalose dihydrate, 10% w/v PEG2000 and 5% w/v graphite, as well as the same formulation utilizing 10% PEG2000 alone. Only low-level expression of pJL1-sfGFP was observed from these reactions in resuspension.

Evaluating these results, CFPS pellets containing pJL1-sfGFP without excipients (except for the previously described cryoprotectants) were fabricated to validate expression in pellets. This is shown in FIG. 5C and FIG 5D. Attempts at fabricating pellets out of the formulation of molecular grade ultrapure water, cell extract, pJL1-sfGFP DNA, and cryoprotectants were unsuccessful as the lyophilized material was observed to be too brittle to be pressed into pellets.

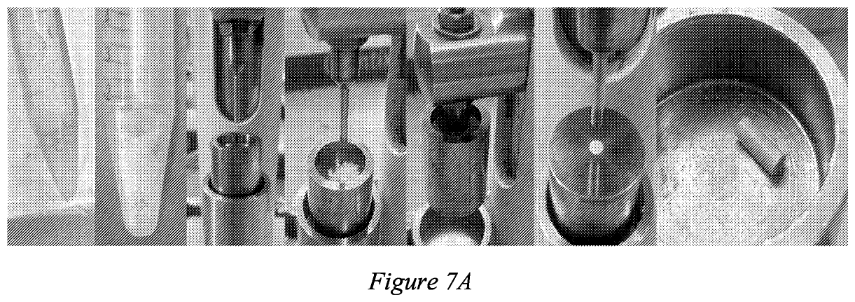

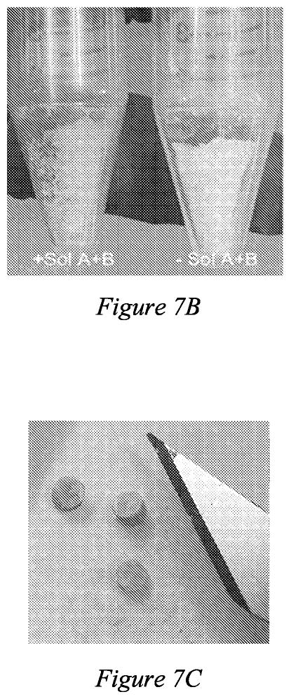

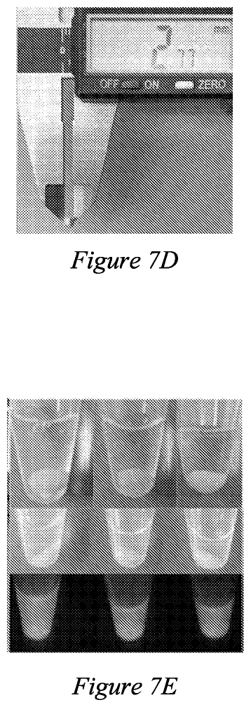

As shown in FIGS. 7A-7E, a solution to this problem was the integration of the energy and cofactor reagents into the lyophilization mixture, which, was observed to increase the cohesiveness of the lyophilized material, making it suitable for pellet fabrication. Testing these pellets by depositing material onto μPADs pJL1-sfGFP expression was observed from the pellet material at the level of the aqueous positive controls. In parallel, the expression of pellets containing 800 μL CFPS reactions utilizing molecular grade ultrapure water, cell extract, pJL1-sfGFP DNA, cryoprotectants, and a decreased concentration of 2.5% PEG2000, as well as the same formulation including the energy and cofactor reagents in the lyophilization mixture were observed and shown in FIG. 5E. Expression was observed to be at the level of the aqueous positive control in the trial without energy and cofactor reagents in the lyophilized mixture and observed to be increased ˜9% compared to the aqueous positive control in the treatment including these reagents in the lyophilization mixture. These results validated that pJL1-sfGFP could be expressed at the level of aqueous controls in a pellet platform and that high-level expression of pJL1-sfGFP in pellets could be maintained with the integration of material excipients. After these expression validation experiments, material metrics testing was conducted to ascertain optimal concentrations of the excipients before moving forward with more expression experiments.

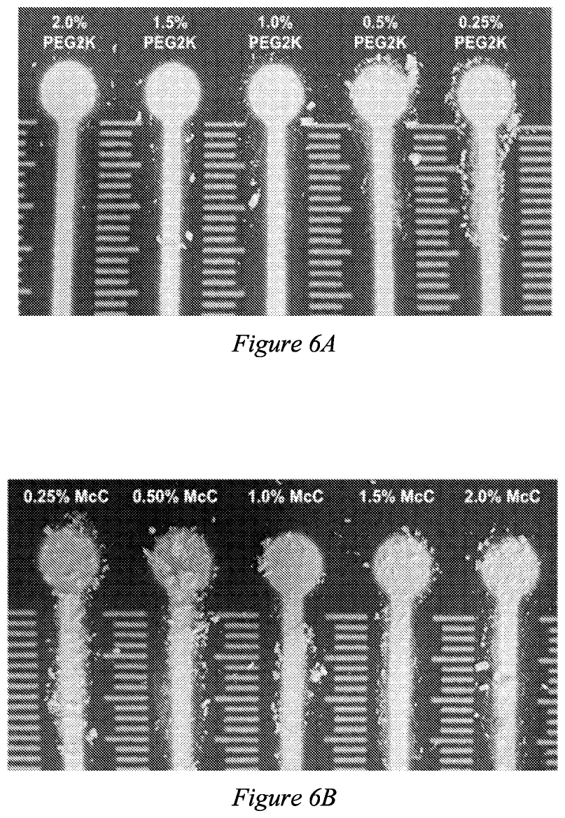

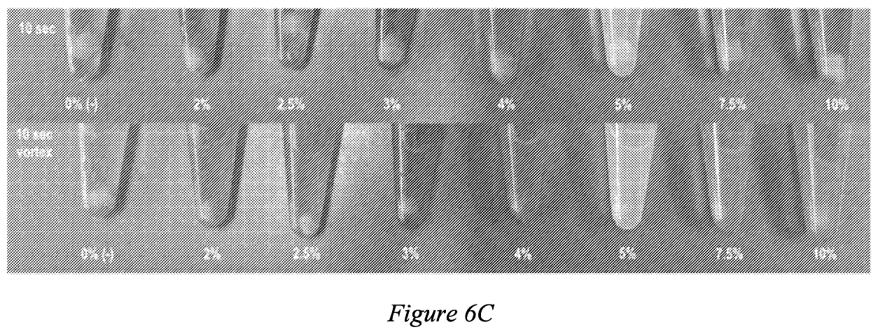

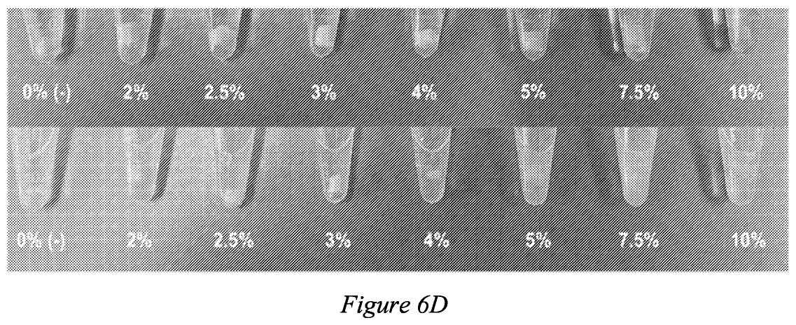

Evaluating the data from the previous expression experiments, pellets containing either PEG2000 or MCC from 0.25% to 2% concentration were fabricated, as well as pellets containing either SSF or Mg2S from 2% to 10% concentration. Multiple material qualities of the integration of these excipients in pellets were evaluated including differences in the fabrication of pellets; differences in mass, length, and diameter of pellets; capacity for deposition of pellet material onto μPADs; and capacity for resuspension of pellets in rehydration solution. The results are shown in FIGS. 6A-6C. It was observed that in the PEG2000 and MCC trials, mass was mostly conserved from lyophilized material to fabricated pellet, with a ˜0.70 mg difference in mass across all trials. Fabrication of the pellet through the pellet press was observed to be uniform through the MCC trials while pressing was notably more difficult with the increase in PEG2000 concentration that was previously observed. Tests of deposition of pellet material onto μPADs demonstrated that the mass of pellet material deposited was observed to decrease with increasing PEG2000 concentration, from >3 mg deposited at 0.25% PEG2000 to <1 mg deposited at 2% PEG2000. The results are shown in FIG. 6A. The opposite trend was observed in the MCC trial, the results of which are shown in FIG. 6B, where the mass of pellet material deposited was observed to increase with increased MCC concentration, from >3 mg deposited at 0.25% MCC to >6 mg deposited at 2% MCC. In resuspension trials, pellets containing 0% (negative control) to 10% SSF or Mg2S were resuspended in water, and rehydration capacity was observed. As shown in FIG. 6C and FIG. 6D, SSF and Mg2S pellets were both observed to resuspend more readily at concentrations of 4% and above after 10-second pulse vortexing.

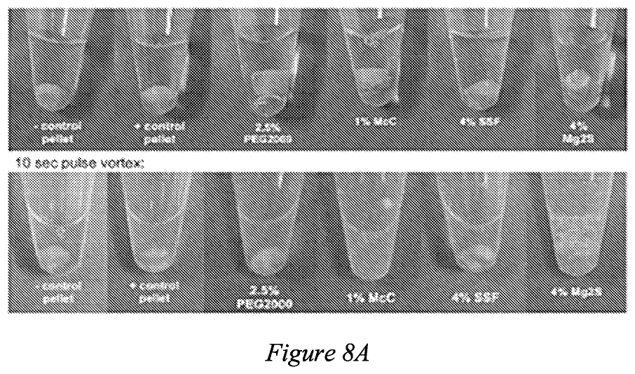

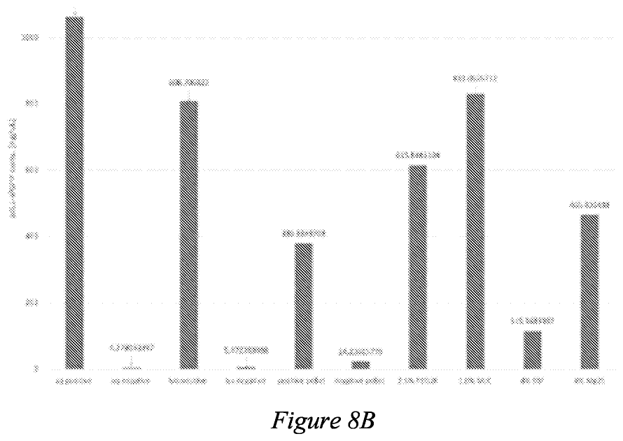

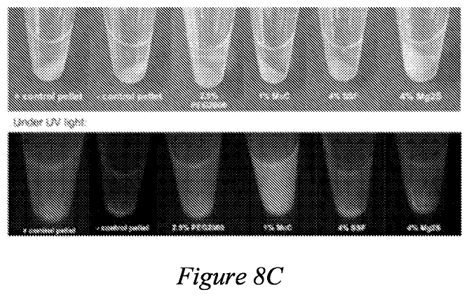

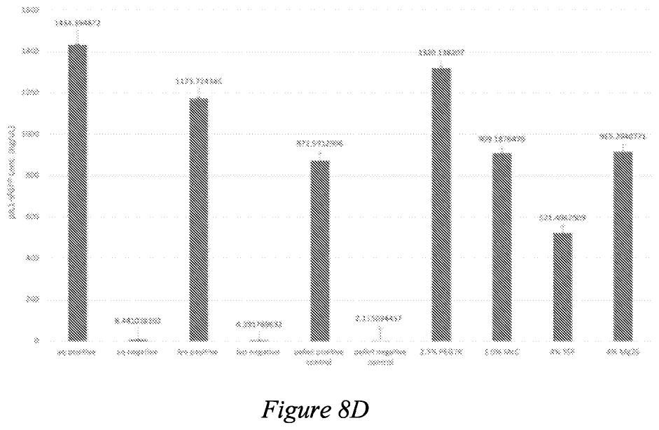

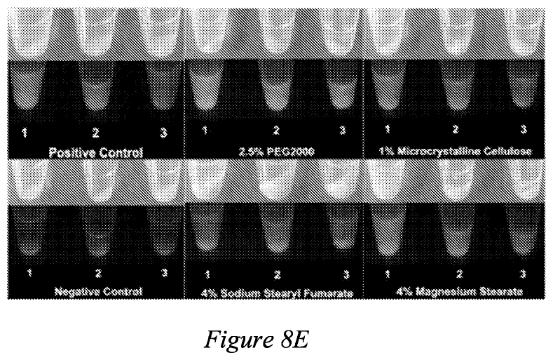

Evaluating the data from these material metrics experiments, experiments were then designed to utilize the optimal concentrations of each excipient in pJL1-sfGFP pellets to test their capacity for resuspension and expression, both as material deposited and rehydrated on μPADs and as resuspended pellets. The results are shown in FIGS. 8A-8E. The optimal concentrations tested were 2.5% PEG2000, 1% MCC, 4% SSF, and 4% Mg2S. The PEG2000 and MCC optimal concentrations were chosen because these concentrations were observed to be only nominally detrimental to pJL1-sfGFP expression in CFPS tests in comparison to positive controls. The SSF and Mg2S optimal concentrations were chosen because these concentrations were observed to be optimal for resuspension without a detrimental decrease in pJL1-sfGFP expression. After a 10-second pulse vortex, the 1% MCC pellet was observed to nearly fully resuspend while the 4% SSF and 4% Mg2S pellets were observed to be partially resuspended.

As shown in FIG. 8A, the 2.5% PEG2000 and control pellets were observed to be only slightly partially resuspended after the 10-second pulse vortex.

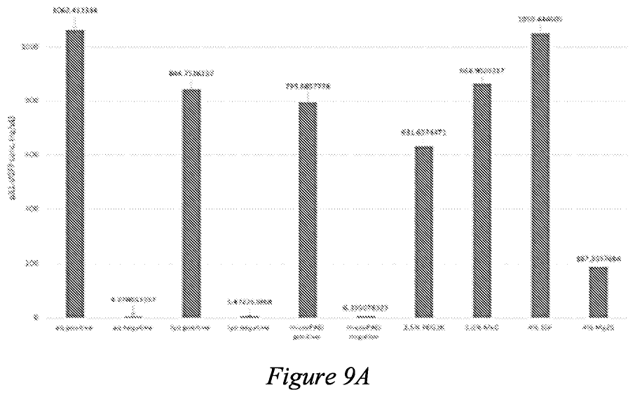

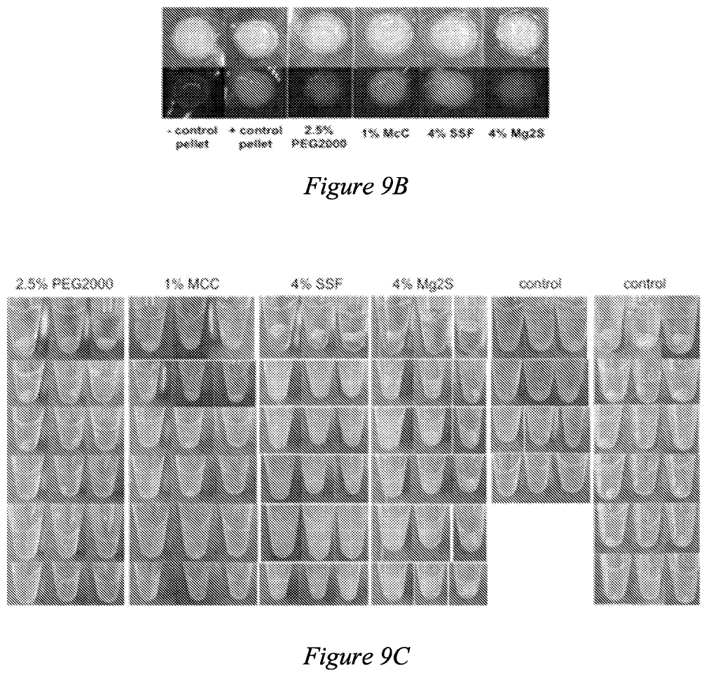

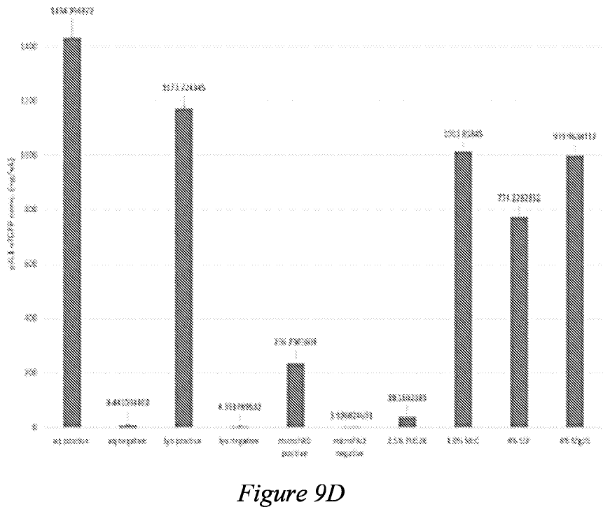

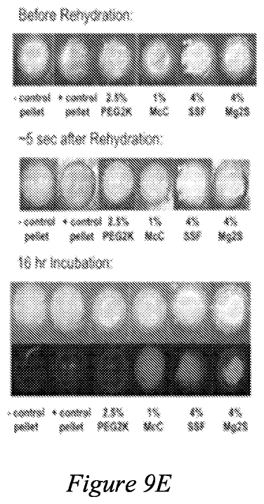

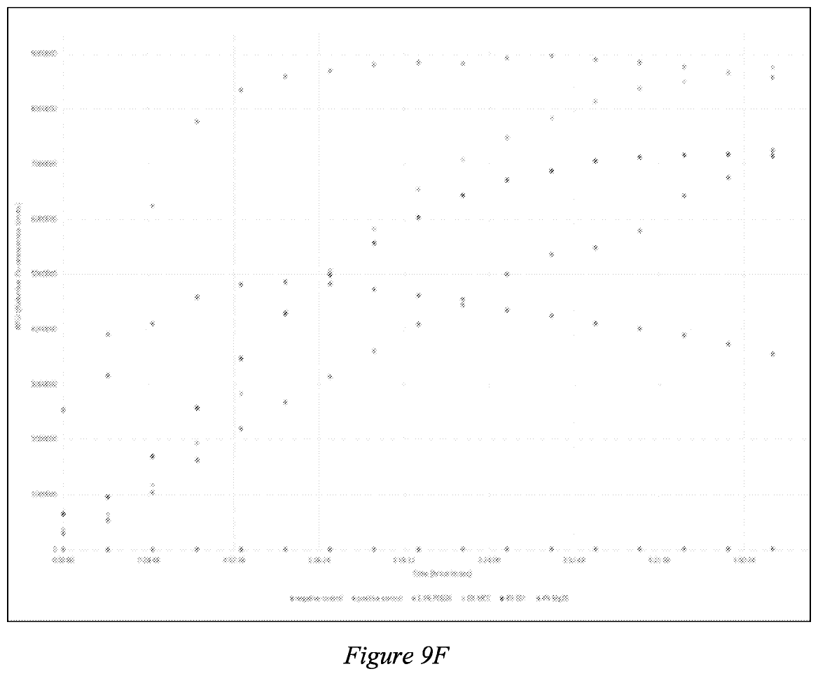

After 14 hr incubation at 37° C. and as shown in FIG. 8B and FIG. 8C, expression of resuspended pellets was observed to be the highest in the 1% MCC trial, nominally above the level of the lyophilized positive control. The other trials were observed to express pJL1-sfGFP at a lower level compared to the lyophilized positive control, with the 2.5% PEG2000 at ˜76%, 4% Mg2S at ˜57%, and 4% SSF at ˜14% of the lyophilized positive control. All resuspended pellet trials except for the 4% SSF trial were observed to have higher expression than the positive control pellet at ˜47% that of the lyophilized positive control. The results are shown in FIGS. 9A-9E. More particularly, as shown in FIG. 9A and FIG. 9B, expression of pellet material rehydrated on μPADs displayed markedly different results with the positive control pellet trial displaying expression at the level of the lyophilized positive control. The 1% MCC and 4% SSF trials were observed to display expression higher than the lyophilized positive control, at ˜7% and ˜30% higher, respectively. The 4% SSF trial in particular was observed to be at the level of the aqueous positive control, a markedly different performance than in the resuspended trial. The 2.5% PEG2000 and 4% Mg2S trials displayed expression below the level of the lyophilized positive control, at ˜78% and ˜23% respectively. The 1% MCC and 2.5% PEG2000 trials were observed to show the greatest similarity in expression between pellet resuspension and μPAD trials, possibly indicating more homogeneity in their pellet fabrication than the 4% SSF and 4% Mg2S trials, resulting in more uniform expression in both resuspension and deposition formats. Evaluating the results, it was determined that further experiments were required to both validate the results of the previous experiment and to answer new questions that arose in the analysis of the results.

The next series of experiments would replicate the one previously described with the addition of segmentation of the pellets into three pellet segments per trial using a scalpel, as well as 5 rounds of 10-second pulse vortex cycles to fully resuspend the pellet segments in rehydration solution before incubation. The rationale for these additions was, in segmentation, to ascertain the homogeneity of reaction components of the pellets, and in vortexing, to more clearly elucidate the pellet segments' capacity for resuspension. As shown in FIG. 9C, in resuspension it was observed that the 2.5% PEG2000 segments nearly completely resuspend in water after 3 rounds of 10-second pulse vortexing, completely resuspending in solution after 5 rounds. The 1% MCC segments were observed to nearly completely resuspend in water after 2 rounds of 10-second pulse vortexing, completely resuspending in solution after 5 rounds. The 4% SSF and 4% Mg2S segments were both observed to substantially break up after a single round of 10-second pulse vortexing, however, remaining at least partially intact after 5 rounds. This result could be in part due to the hydrophobic characteristics of the excipients breaking from the rest of the pellet material, leaving the CFPS components intact. Surprisingly, the positive control pellet segments were observed to partially resuspend quickly in water without pulse vortexing and completely resuspend after 3 rounds of 10-second pulse vortexing. This is uncharacteristic compared to other experiments and is likely due to observed difficulties in pellet fabrication and segmentation of the lyophilized material without material excipients, such as observed stickiness in some observed pellet fabrication. The negative control pellet segments remained partially intact after 4 rounds of 10-second pulse vortexing, however, were observed to nearly completely resuspend after the 5th round.

Observing the expression of resuspended pellet segments after 14 hr incubation at 37° C., all treatments demonstrated improvement in expression compared to the full pellet resuspension of the previously described experiment (see, e.g., FIG. 8D and FIG. 8E). The 2.5% PEG2000 trial performed the best, with a segment expression average ˜51% higher than the pellet positive control, ˜16% higher than the lyophilized positive control, and ˜92% of the expression of the aqueous positive control. The 1% MCC and 4% Mg2S trials performed similarly well to each other, at ˜80% of the lyophilized positive control and nominally outperforming the pellet positive control at ˜4-5% higher expression. As observed in the previous full pellet resuspension experiment, the 4% SSF trial was the lowest performer at ˜46% of the expression of the lyophilized positive control and ˜59% of the expression of the pellet positive control. The pellet positive control itself performed under both the aqueous and lyophilized positive controls, at ˜61% and ˜77% respectively. The error between the pellet segments' expression was lowest in the 2.5% PEG2000 trial and fairly low overall, demonstrating homogeneity of reaction components across the pellets. Qualitatively, pJL1-sfGFP expression was much more visible than in the full pellet resuspension, confirming the quantitative results and showcasing the potential benefit of pellet segmentation. pJL1-sfGFP expression on μPADs was observed to improve in the 1.0% MCC and 4% Mg2S trials, at ˜70% the expression of aqueous positive control and ˜90% the expression of the lyophilized positive control. These results are shown in FIG. 9D.

The 4% SSF trial was observed to show good but decreased expression compared to the previous experiment, at ˜68% of the lyophilized positive control. The pellet μPAD positive control and 2.5% PEG2000 trials were observed to have very low expression, at ˜21% and 3% the expression of the lyophilized positive control respectively. This result is likely due to experimental error in either deposition or rehydration of the pellet material on the μPADs. The 4% SSF trial was observed to be the most optimal for pellet material deposition onto μPADs while the 1% MCC trial was observed to have the most optimal μPAD visual appearance and signal. Evaluating the results, it was noted that, while different treatments demonstrated high performance, no one excipient was optimal in all of the experimental categories tested. Next, combinations of the excipients were tested using the protocols described in the previous examples to test excipient combination pellet segments' capacity for resuspension, deposition, and expression.

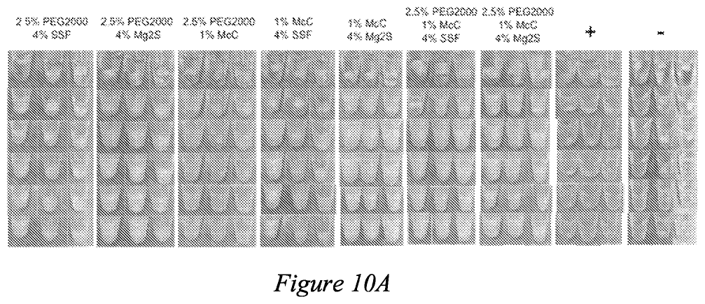

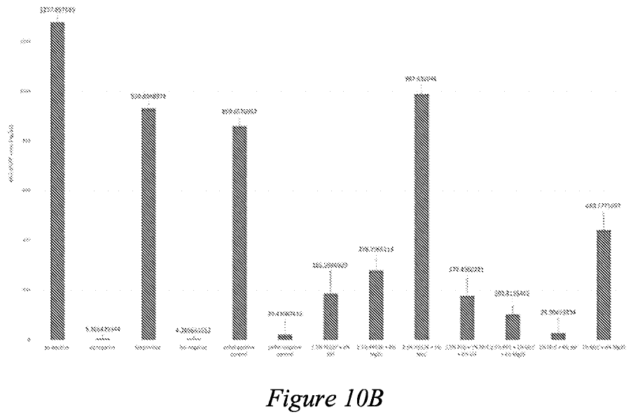

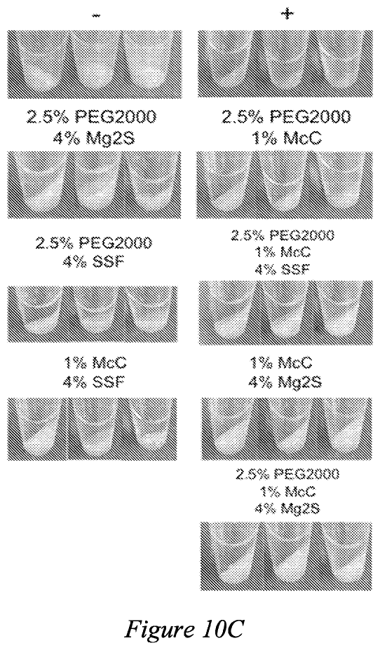

Taking a combinatorial approach, the chosen treatments included: 2.5% PEG2000+4% SSF; 2.5% PEG2000+4% Mg2S; 2.5% PEG2000+1% MCC; 1% MCC+4% SSF; 1% MCC+4% Mg2S; 2.5% PEG2000+1% MCC+4% SSF; 2.5% PEG2000+1% MCC+4% Mg2S; along with positive and negative control pellets. As with the previous experiments, all pellet formulations consisted of an 800 uL CFPS reaction utilizing molecular grade ultrapure water, BL21*DE3 cell extract, and pJL1-sfGFP DNA with 0.4% PEG8000, 20 mM Trehalose Dihydrate cryoprotectants, along with the aforementioned treatments. As shown in FIG. 10A, in segment resuspension, the 2.5% PEG2000+4% SSF treatment was observed to mostly resuspend across segments, although full resuspension could not be achieved after 5 rounds of 10-second pulse vortexing. The 2.5% PEG2000+4% Mg2S treatment was observed to fully resuspend across segments after 2 rounds of 10-second pulse vortexing. The 2.5% PEG2000+1% MCC treatment was observed to nearly fully resuspend in two of the three segments after 2 rounds of 10-second pulse vortexing and completely resuspend after 4 rounds. The 1% MCC+4% SSF treatment was observed to nearly fully resuspend in two of the three segments after 3 rounds of 10-second pulse vortexing and completely resuspend after 4 rounds. The 1% MCC+4% Mg2S treatment was observed to fully resuspend across segments after 2 rounds of 10-second pulse vortexing. The 2.5% PEG2000+1% MCC+4% SSF treatment was observed to nearly fully resuspend after 2 rounds of 10-second pulse vortexing and completely resuspend after 3 rounds. The 2.5% PEG2000+1% MCC+4% Mg2S was observed to fully resuspend in two of the three segments after 3 rounds of 10-second pulse vortexing, however, one of the segments remained slightly intact after 5 rounds. Both the positive and negative control pellets were observed to mostly resuspend after 5 rounds of 10-second pulse vortexing, however, only one positive control segment was observed to fully resuspend.

pJL1-sfGFP expression of resuspended pellet segments was observed after 16 hr incubation at 37° C. As shown in FIG. 10B and FIG. 10C, the 2.5% PEG2000+1% MCC treatment demonstrated the highest average segment expression at ˜15% above the level of the positive pellet control, ˜6% above the level of the lyophilized positive control, and ˜77% of the expression of the aqueous positive control. The other treatments demonstrated low-level segment average expression, all below ⅓ of the expression of the pellet positive control with the exception of the 1% MCC+4Mg2S treatment, which displayed ˜51% of the expression of the pellet positive control. The pellet positive control segment average expression was observed to be ˜92% of the expression of the lyophilized positive control and ˜67% of the aqueous positive control. The 2.5%PEG2000+1% MCC treatment was the only pellet treatment to demonstrate error lower than the level of the positive controls, demonstrating the treatments' ability to maintain high-level expression while maintaining homogeneity of reaction components above that of the other treatments. Taken cumulatively, these results demonstrate that many of the combinatorial treatments could improve pellet resuspension but at the cost of pJL1-sfGFP expression. The notable exceptions to this, the 2.5%PEG2000+1% MCC and 1% MCC+4% Mg2S treatments, demonstrated improved resuspension of pellets while maintaining pJL1-sfGFP expression.

### Example 6. CRISPR-SpCas9

Clustered Regularly Interspersed Short Palindromic Repeats (CRISPR) is a method of gene editing derived from prokaryotic DNA sequences that function in the defense of prokaryotes from viruses such as bacteriophages. CRISPR makes use of the Cas endonuclease, an enzyme that cleaves specific nucleotides within double-stranded DNA, as well as guide RNA (gRNA) complementary to a specific DNA sequence that functions to lead the CRISPR-Cas complex to the intended gene target. The CRISPR-Cas gene-editing system is widely used within industry and academia for a range of biotechnological applications, so it was reasoned that integrating this technology into the pellet platform would provide a prime test case for the potential of the platform to support complex and useful biotechnologies. For the CRISPR-Cas system, a CRISPR system utilizing SpCas9, a popular Cas endonuclease derived from Streptococcus pyogenes, with gRNA complementary to the pY71-mRFP1 gene expressing Red Fluorescent Protein (RFP) was selected. The CRISPR-SpCas9 system functions to quench pY71-mRFP1 expression, in which the presence of the associated CRISPR-SpCas9 components results in a sharp decrease in fluorescence. To successfully integrate this system into the pellet platform two main technical issues needed to be addressed. These factors were first, prototyping expression of pY71-mRFP1 along with the CRISPR-SpCas9 system in CFPS, and second, integrating and expressing pY71-mRFP1 in the pellet platform.

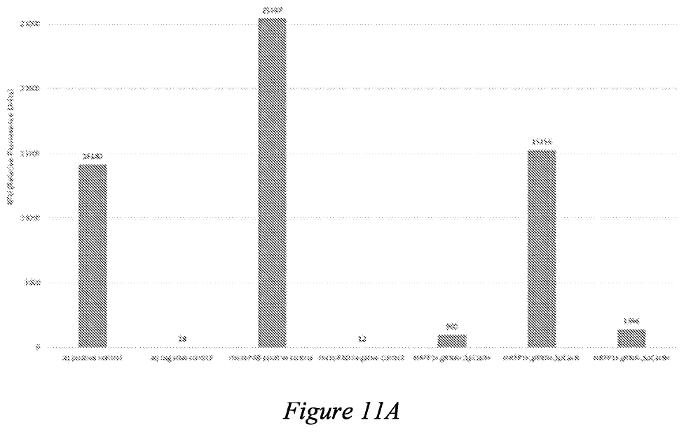

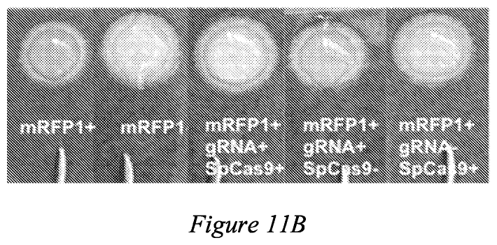

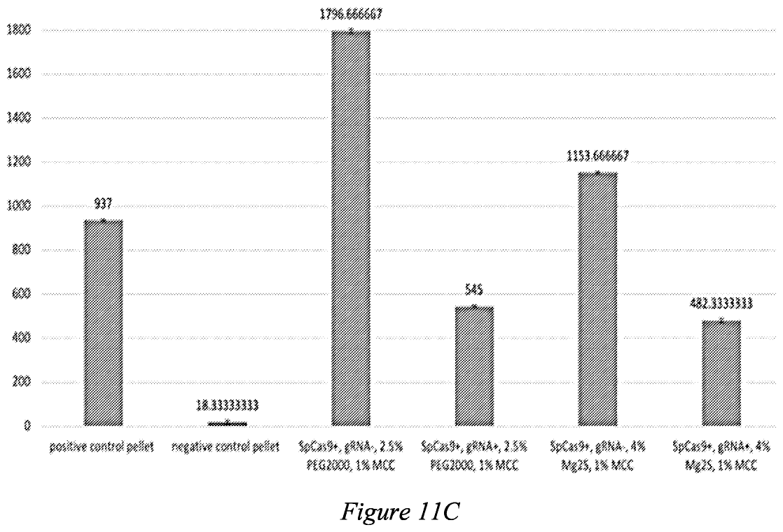

Initial CFPS reactions expressing the CRISPR-SpCas9 system consisted of the previously described CFPS reaction components: molecular grade ultrapure water, BL21*DE3 cell extract, energy and cofactor reagents, and a number of DNA constructs depending on the treatment. The DNA constructs utilized were pY71-mRFP1 expressing RFP, SpCas9 DNA expressing the SpCas9 endonuclease, and mRFP1 gRNA DNA expressing the gRNA specific to the mRFP1 gene. the treatments included: mRFP1+, gRNA+, SpCas9+; mRFP1+, gRNA+, SpCas9−; mRFP1+, gRNA−, SpCas9+; and the mRFP1 positive and negative controls containing neither SpCas9 nor gRNA DNA. The results are shown in FIG. 11A, FIG. 11B, and FIG. 11C. More particularly, as shown in FIG. 11A and FIG. 11B, and as anticipated, both the mRFP1 positive control and mRFP1+, gRNA+, SpCas9-treatment demonstrated high-level mRFP1 expression at 11194.67 and 18870.44 Relative Fluorescence Units (RFU) respectively. The mRFP1+, gRNA+, SpCas9+ and mRFP1+, gRNA−, and SpCas9+ were observed to have similarly quenched mRFP1 expression at 373.78 and 512.33 RFU respectively. These values represented a decrease in mRFP1 expression compared to the positive control of >95% and served to confirm that SpCas9 was cleaving the mRFP1 DNA, quenching expression as expected. The large drop in expression in both treatments however despite one possessing the mRFP1 gRNA and the other was not expected, as the mRFP1+, gRNA−, SpCas9+ treatment should have seen a modest drop in expression but not a near-total quenching of mRFP1 expression. Parallel aqueous CFPS tests on μPADs validated these results with a similar trend.

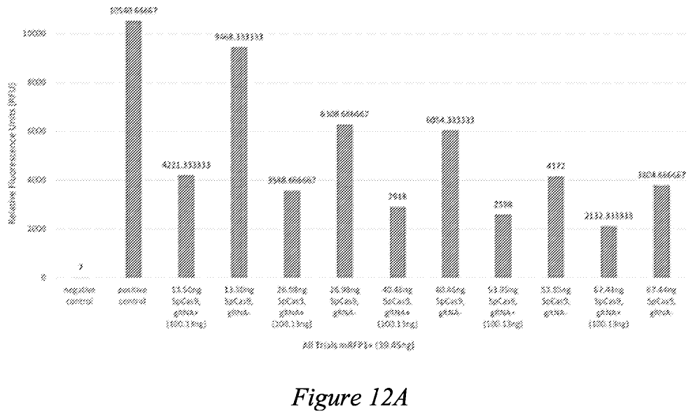

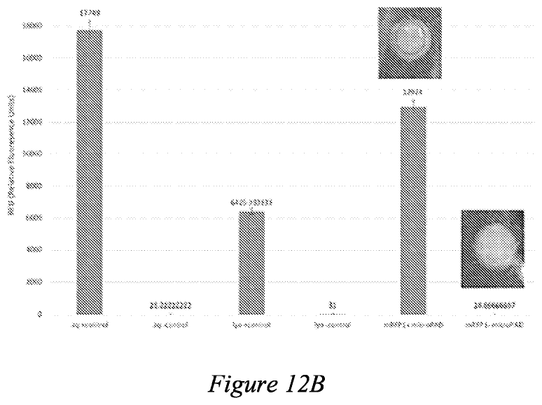

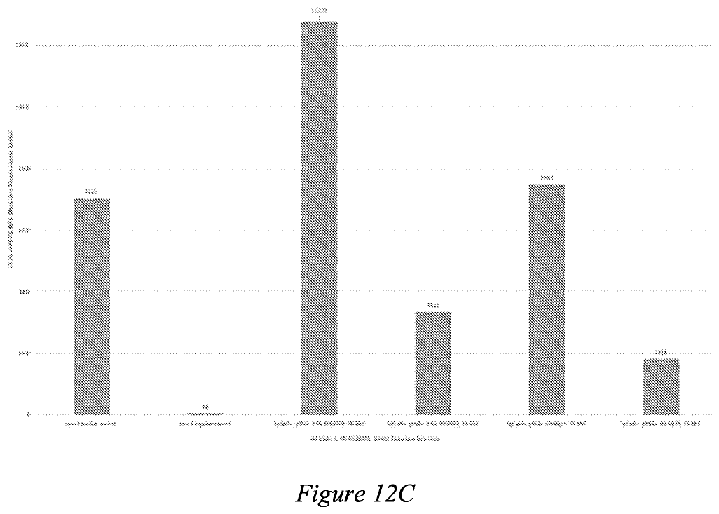

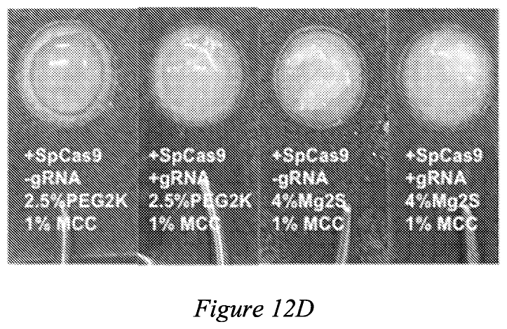

It was hypothesized that the CFPS mixture in these treatments might be oversaturated with SpCas9 DNA, which may have caused the endonuclease to cleave DNA non-specifically. To test this hypothesis a SpCas9 titration CFPS was performed in which the treatments that were positive for gRNA DNA were kept at a constant concentration of 100.13 ng for all trials. The results are shown in FIG. 12A. For both the mRFP1 gRNA DNA positive and negative treatments, SpCas9 DNA concentration was increased in 5 dual mRFP1 gRNA DNA positive/negative treatments from 13.50 ng to 67.44 ng. For all test trials, the concentration of pY71-mRFP1 was kept at a constant 39.45 ng. After 14 hr incubation at 37° C., it was observed that there was a clear increase in SpCas9 activity with the addition of mRFP1 gRNA DNA, with the distinction in pY71-mRFP1 expression between mRFP1 gRNA DNA positive and negative treatments appearing more apparent with the decrease in SpCas9 DNA concentration. This was most apparent in the 13.50 ng SpCas9 DNA treatments, where the mRFP1-gRNA DNA positive treatment was observed to have a pY71-mRFP1 expression of 4221.33 RFU compared to the mRFP1-gRNA DNA negative treatment with 9468.33 RFU. These values represent a difference in pY71-mRFP1 expression of˜55%, clearly demonstrating the increased endonuclease function of SpCas9 with the introduction of mRFP1-gRNA. The 13.50 ng SpCas9 DNA, mRFP1-gRNA DNA negative trial also displayed modestly decreased expression compared to the positive control at ˜90% of the positive control expression, demonstrating the expected drop in expression without the presence of mRFP1-gRNA.

With the expression of the CRISPR-SpCas9 system substantially improved in CFPS, the next step was to validate the integration of pY71-mRFP1 expression into the pellet platform. the pellet mixture consisted of the same formulation previously described for pJL1-sfGFP pellet expression: molecular grade ultrapure water, BL21*DE3 cell extract, energy and cofactor reagents, cryoprotectants 0.4% PEG8000 and 20 mM Trehalose Dihydrate, and pY71-mRFP1 DNA. pY71-mRFP1 positive and negative pellet material was deposited on μPADs and incubated for 14 hr at 37° C. After incubation, the pY71-mRFP1 positive μPAD was observed to display clear red fluorescence with a high visual signal while the negative μPAD showed no such signal. As shown in FIG. 12B, the pY71-mRFP1 positive μPAD was observed to have an expression value of 12974 RFU, a greater than 2-fold increase over the lyophilized positive control at 6426.33 RFU. This result demonstrated that pY71-mRFP1 could be expressed via CFPS reaction in the pellet platform.

### Example 7. Analysis of CFPS Expression of the CRISPR-SpCas9 System

With the two main factors necessary for integration validated, the next step was to test the CFPS expression of the CRISPR-SpCas9 system in the pellet platform. The experiment was designed with four treatments: two containing the previously described pellet formulation along with 2.5% PEG2000+1% MCC and the other two containing 4%Mg2S+1% MCC, the optimal excipient combinations previously validated for the expression of pJL1-sfGFP; one pellet of each excipient treatment consisted of mRFP1-gRNA DNA while the other two were negative for mRFP1-gRNA DNA. Lastly, all test pellets consisted of pY71-mRFP1 and SpCas9 DNA. The pellets were tested via deposition onto μPADs as well as full-pellet resuspension in water and incubated for 14 hrs at 37° C.

As shown in FIG. 12C and FIG. 12D, after incubation, the SpCas9+, mRFP1-gRNA−, 2.5% PEG2000 +1% MCC treatment was observed to display the highest level of pY71-mRFP1 expression at 12779 RFU, compared to the SpCas9+, mRFP1-gRNA+, 2.5% PEG2000+1% MCC treatment which displayed a quenched expression of 3337. The same trend was observed in the 4%Mg2S+1% MCC treatments with the SpCas9+, mRFP1-gRNA−, 4%Mg2S+1% MCC treatment displaying expression slightly above the level of the pellet positive control at 7483 RFU, compared with the SpCas9+, mRFP1-gRNA+, 4%Mg2S+1% MCC treatment displaying a quenched expression of 1814. The decrease in fluorescence between the gRNA positive and negative trials of ˜74% in the 2.5% PEG2000+1% MCC trials and ˜76% 4%Mg2S+1% MCC trials suggests that CRISPR-SpCas9 activity, guided by the mRFP1-gRNA, resulted in a drop in expression of pY71-mRFP1. As shown in FIG. 11C, in the full pellet resuspension, expression was decreased compared to μPAD expression but followed the same trend in all test treatments. As in the μPAD treatment, the SpCas9+, mRFP1-gRNA−, 2.5% PEG2000+1% MCC treatment was observed to display the highest level of pY71-mRFP1 expression at 1796.67 RFU, compared to the SpCas9+, mRFP1-gRNA+, 2.5% PEG2000+1% MCC treatment which displayed a quenched expression of 545 RFU. The same trend was observed in the 4%Mg2S+1% MCC treatments with the SpCas9+, mRFP1-gRNA−, 4%Mg2S+1% MCC treatment displaying expression slightly above the level of the pellet positive control at 1153.67 RFU, compared with the SpCas9+, mRFP1-gRNA+, 4%Mg2S+1% MCC treatment displaying a quenched expression of 482.33 RFU. The decrease in fluorescence between the gRNA positive and negative trials of ˜70% in the 2.5% PEG2000+1% MCC trials and ˜58% 4%Mg2S+1% MCC trials suggests that like in the μPAD test the CRISPR-SpCas9 activity, guided by the mRFP1-gRNA, resulted in a drop in expression of pY71-mRFP1. While the μPAD signal was much more visible than resuspended pellets in this experiment, fluorescence quantification confirmed that CRISPR-SpCas9 activity followed the same trends in both resuspension and deposition formats. Most importantly, this experiment validated the ability of the pellet platform to express through CFPS and support complex biotechnologies like CRISPR-Cas.

### Example 8. Developing μPADs for CFPS Reactions

μPADs, as described earlier, are paper-based microfluidic devices that have been demonstrated to support biochemical reactions. To develop devices to successfully support CFPS reactions, the validation of several key factors were identified: 1), the capacity to support CFPS reactions during incubation without evaporation of the sample solution, 2) the strength of the visual signal of the sample on μPADs for qualitative validation, and 3) the capacity to fully resuspend and quantify pellet-deposited samples on μPADs.

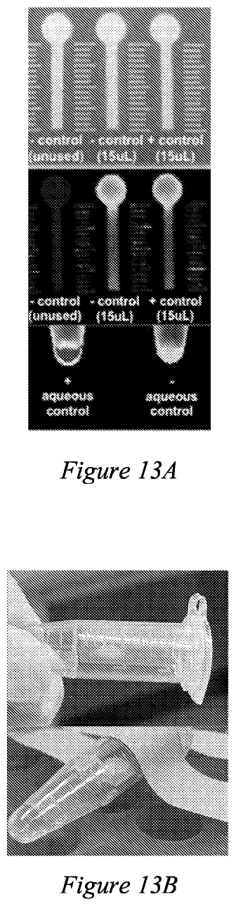

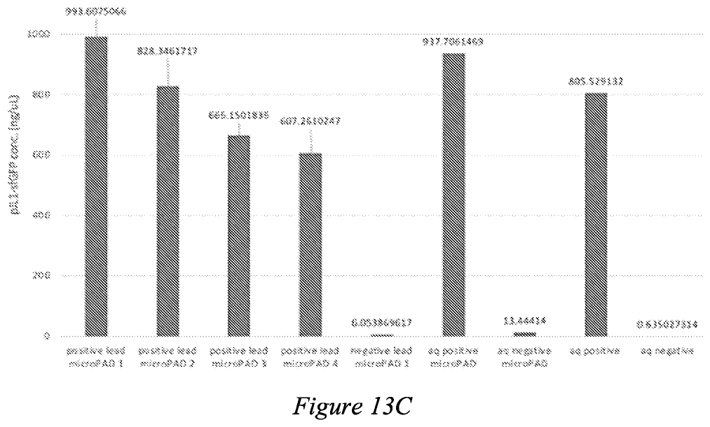

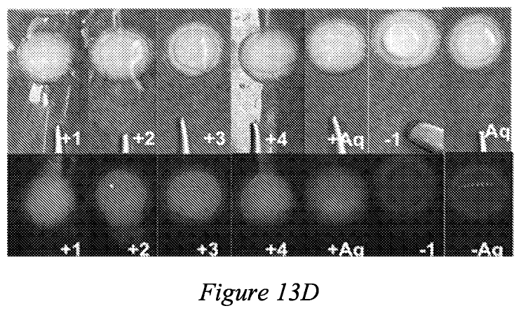

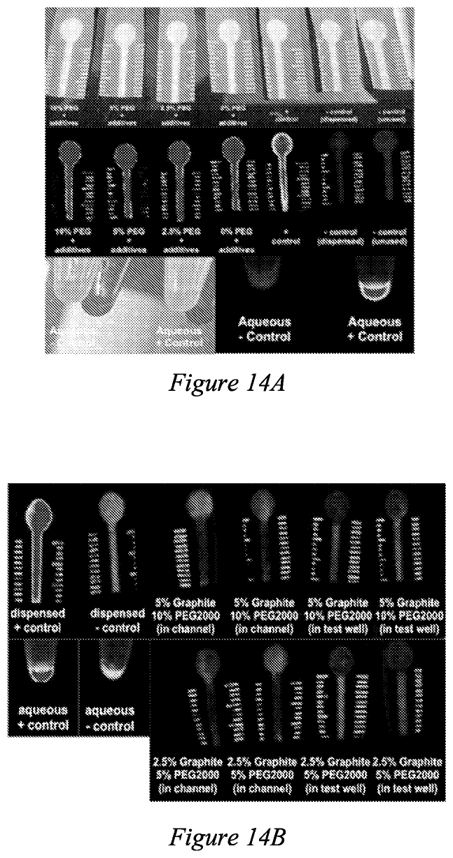

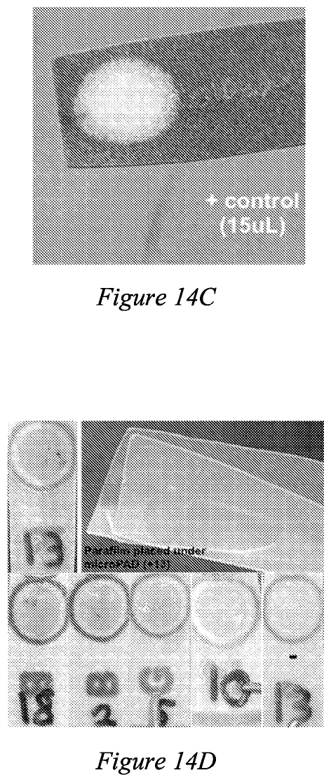

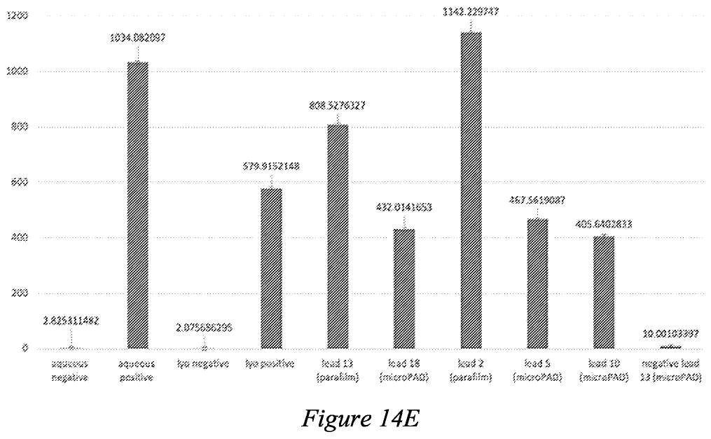

In the initial CFPS experiments performed on μPADs, both aqueous and pellet depositions suffered from issues with the evaporation of the rehydration solution off of the μPADs. It was hypothesized that perhaps by laminating the sample channel of the PAD it would be possible to eliminate evaporation as a concern. After fabricating μPADs with open sample wells for sample deposition with laminated sample channels, a simple experiment was run dispensing 15 uL of pJL1-sfGFP positive and negative cell-free reactions into parallel laminated μPADs, then incubated them for 14 hr at 37 C. As shown in FIG. 13A, after incubation no visual signal of expression was observed, with positive and negative treatments appearing identical. Under UV imaging, a definable difference between the sample well and channel of the negative and positive μPADs as well as between used and unused μPADs was observed, showing that the reaction solution was able to travel through the length of the channel but did not express pJL1-sfGFP. It was hypothesized that this could have been due to a lack of oxygen availability in the laminated channel, as CFPS reactions require oxygen to function.

Reverting to the unlaminated μPADs, the next step was to incubate the μPADs in Eppendorf tubes to prevent evaporation. This is shown in FIG. 13B. 50μL of nanopure water was also added to the bottom of the Eppendorf tubes to increase the vapor pressure in the tube to further prevent evaporation of the μPAD samples. This solution was validated in μPAD CFPS experiments and standardized for all further experimentation using μPADS.

Focusing next on visual signal strength of the CFPS reactions expressing pJL1-sfGFP on μPADs, initial experiments were run testing aqueous and pellet deposition of CFPS reactions containing varying combinations of graphite and PEG2000 in pellet deposition and PEG2000 alone in aqueous deposition. In the aqueous dispensed tests, it was found that the sfGFP fluorescence signal was, while clearly visible under UV imaging, barely distinguishable from negative controls in visible light. These results are shown in FIG. 14A. Further, as shown in FIG. 14B, in the pellet deposition tests, no sfGFP fluorescence signal was observed under UV imaging or visible light. This was likely due to the mixture of graphite and PEG2000, which was previously observed to be detrimental to pJL1-sfGFP expression. It was also observed, however, that graphite would cloud any fluorescence signal on the μPAD. In view of these results, it was determined that the test region of the μPAD needed to have a smaller surface area to increase the visual signal of sfGFP fluorescence and that graphite would need to be phased out of pellet formulations so as not to cloud visual signal.

For the design of the next iterations of μPAD, the test channel was removed and in its place was used a single circular well surrounded by a hydrophobic barrier where the CFPS reaction would take place. As shown in FIG. 14C, in aqueous deposition tests, these μPADS were demonstrated to be able to both support CFPS reactions and provide a higher signal than the previous iteration. In pellet-deposited experiments, although CFPS reactions were able to be performed and sfGFP signal did increase, pellet material was observed to be not completely rehydrating on the μPADs and it was difficult to collect reaction solution after incubation for subsequent quantification. It was hypothesized that the reaction solution was, during incubation, leaking through the μPADs. It was further posited that this leakage was due to the wax hydrophobic barriers of the μPAD breaking down over the course of the reaction, which was believed to be the cause of the pellet material not rehydrating fully during incubation. In expression tests shown in FIG. 14D and FIG. 14E, this hypothesis was tested by placing parafilm under the backsides of the μPADs during incubation. After incubation it was observed that the parafilm was able to collect reaction solution, confirming the leakage hypothesis. Quantification of the reaction solution collected from parafilm showed that expression levels reached the levels of the positive controls, validating that rehydrated pellet material could be expressed at a high level on μPADs.

To solve the problem of leakage the μPADs were laminated entirely except for the well region where the CFPS reaction would take place. The rationale was that the laminate layer would prevent the reaction solution from leaking, allowing the pellet material to be fully rehydrated on the μPADs. As shown in FIG. 13C and FIG. 13D, in expression tests using these μPADs it was observed that leakage was prevented by the laminate layer, and the pellet material deposited on the μPADs was fully rehydrated. Quantification of the reaction solutions demonstrated pJL1-sfGFP expression at the level of the positive controls, validating the μPADs' and pellets' ability to support high-level CFPS reactions.

The embodiments being thus described, it will be obvious that the same may be varied in many ways. Such variations are not to be regarded as a departure from the spirit and scope of the disclosure and all such modifications are intended to be included within the scope of the following claims.

1. A composition for solid-state storage comprising:
a polymeric cryoprotectant;
a saccharide cryoprotectant;
an emulsifier; and
a lubricating flow agent.

2. The composition of claim 1, wherein the polymeric cryoprotectant comprises a polyethylene oxide polymer, vinylpyrrolidone polymer, polysaccharide, poloxamer, methoxy polyethylene glycol acrylate, polyampholyte, or a combination thereof.

3. The composition of claim 2, wherein the polyethylene oxide polymer comprises polyethylene glycol (PEG) having between 200 and 80000 ethylene glycol residues.

4. The composition of claim 1, wherein the saccharide cryoprotectant comprises a sugar alcohol, monosaccharide, disaccharide, oligosaccharide, polysaccharide, or a combination thereof.

5. The composition of claim 1, wherein the saccharide cryoprotectant comprises glucose, galactose, fructose, ribose, lactose, sucrose, lactulose, trehalose, melibiose, glycerol, sorbitol, mannitol, maltitol, a starch, dextrin, dextran, cellulose, maltose, methylcellulose, hemicellulose, pectin, gelatin, glycogen, chitin, heparin, maltodextrin, a sugar alcohol, or a combination thereof.

6. The composition of claim 5, wherein the saccharide cryoprotectant comprises trehalose dihydrate, anhydrous dextrose, anhydrous glucose, anhydrous crystalline maltose, or a combination thereof.

7. The composition of claim 1, wherein the emulsifier comprises microcrystalline cellulose, defibrillated microcrystalline cellulose, microfibrillated cellulose, powdered cellulose, cellulose, cellulose acetate, hydroxypropyl methylcellulose, carboxymethylcellulose, sodium carboxymethylcellulose, ethylcellulose, methylcellulose, hydroxyethylcellulose, hydroxyethylcellulose, hydroxypropylcellulose, silica nanoparticles, carbon nanotubes, or a combination thereof.

8. The composition of claim 1, wherein the lubricating flow agent comprises a fatty acid metal salt, a fatty acid, hydrocarbon, fatty alcohol, fatty acid ester, alkyl sulfate, polymer, silicate, or a combination thereof.

9. The composition of claim 8, wherein the fatty acid comprises oleic acid, butyric acid, lauric acid, palmitic acid, benzoic acid, fumaric acid, stearic acid, or a combination thereof; and wherein the fatty acid comprises a counterion comprising a metal or a metal salt comprising lithium, sodium, potassium, magnesium, calcium, barium, lead, zinc, cadmium, or a combination thereof.

10. The composition of claim 1, wherein the lubricating flow agent comprises a first lubricating flow agent and a second lubricating flow agent.

11. The composition of claim 10, wherein the first lubricating flow agent comprises sodium stearyl fumarate and wherein the second lubricating flow agent comprises magnesium stearate.

12. The composition of claim 1, wherein the composition comprises between about 0.5% to about 10% of the polymeric cryoprotectant, between about 1 mM to about 50 mM of the saccharide cryoprotectant, between about 0.1% to about 10% of the emulsifier; and between about 0.5% and about 20% of the lubricating flow agent.

13. A method of stabilizing a biological system in solid-state storage comprising:
contacting a biological system with the composition of claim 1 to form a mixture;
lyophilizing the mixture; and
forming the mixture into a solid.

14. The method of claim 13, wherein the solid is a powder, flowable powder, agglomerate, flake, granule, pellet, tablet, or pencil.

15. The method of claim 13, wherein the solid can be stored and transported at room temperature (20° C. to 22° C.) and retain at least 80% activity of the biological system.

16. The method of claim 13, further comprising a step of depositing the solid onto a substrate.

17. The method of claim 16, wherein the substrate is a microfluidic chip or a paper-based microfluidic device.

18. A method of using a stabilized biological system comprising:
(i) obtaining a stabilized biological system comprising a biological system, a polymeric cryoprotectant, a saccharide cryoprotectant, an emulsifier, and a lubricating flow agent; wherein the stabilized biological system is a solid;
(ii) optionally depositing the stabilized biological system onto a substrate; and
(iii) contacting the stabilized biological system with a liquid to form a resuspension solution.

19. The method of claim 18, further comprising:
(iv) collecting a biological sample;
(v) reacting the resuspension solution with the biological sample to generate a result; and
(vi) interpreting the result.

20. A paper-based microfluidic device comprising:
a porous substrate comprising paper;
an input channel;
a reaction chamber;
one or more microfluidic channels; and
a non-porous barrier that defines one or more boundaries for the reaction chamber and the one or more microfluidic channels;
wherein the reaction chamber is fluidly connected to the input chamber by the one or more microfluidic channels; and
wherein a surface of the microfluidic device is laminated except for the reaction chamber.
